(19) United States
(12) Patent Application Publication
(10) Pub. No.: US 2025/0027939 A1
(43) Pub. Date: Jan. 23, 2025

(54) AFFINITY PURIFICATION, PROXIMITY-BASED SORTASE LIGATION, AND DETECTION OF PROTEINS WITH PRECURSOR PEPTIDES AND B1 PROTEINS FROM LASSO PEPTIDE BIOSYNTHESIS SYSTEMS

(71) Applicants: THE TRUSTEES OF THE UNIVERSITY OF PENNSYLVANIA, Philadelphia, PA (US); AlphaThera LLC, Philadelphia, PA (US)

(72) Inventors: Andrew TSOURKAS, Bryn Mawr, PA (US); Yi Shu, Paoli, PA (US)

(73) Assignees: THE TRUSTEES OF THE UNIVERSITY OF PENNSYLVANIA, Philadelphia, PA (US); AlphaThera LLC, Philadelphia, PA (US)

(21) Appl. No.: 18/716,935

(22) PCT Filed: Dec. 8, 2022
(86) PCT No.: PCT/US22/81220

§ 371 Date: Jun. 6, 2024

Related U.S. Application Data
(60) Provisional application No. 63/288,483, filed on Dec. 10, 2021.

(51) Int. Cl. G01N 33/569; C07K 14/195; C07K 16/12

(52) U.S. Cl. CPC G01N 33/56911; C07K 14/195; C07K 16/1267; C07K 2319/50; C07K 2319/60; G01N 2333/952

(57) ABSTRACT

The invention relates to the use of precursor peptides and B1 proteins from lasso peptide biosynthesis systems for affinity purification, proximity-based sortase-mediated protein purification and ligation, and detection of fusion proteins. For proximity-based sortase-mediated protein purification and ligation, the invention relates to techniques that link protein purification with conjugation to other agents, including therapeutic agents, imaging agents, or linkers.

### GOVERNMENT INTEREST STATEMENT

This invention was made with government support under grant number CA221374 awarded by the National Institutes of Health. The government has certain rights in the invention.

### FIELD OF THE INVENTION

The invention relates to the use of precursor peptides and B1 proteins from lasso peptide biosynthesis systems for affinity purification, proximity-based sortase-mediated protein purification and ligation, and detection of fusion proteins. For proximity-based sortase-mediated protein purification and ligation, the invention relates to techniques that link protein purification with conjugation to other agents, including therapeutic agents, imaging agents, or linkers.

### BACKGROUND OF THE INVENTION

In many basic science, commercial or clinical applications, it is necessary to label a protein

with a cargo. The cargo can be a biomolecule, a drug, an imaging agent, a chemical moiety (e.g., hapten, click chemistry molecule, etc.) or other compounds. While bioconjugation approaches exist to prepare protein-cargo conjugates, the method used can significantly impact the function of the protein and/or cargo as well as the homogeneity of the resulting conjugate. For example, the number and location of cargo can vary significantly from protein to protein.

Proximity-based sortase-mediated ligation (PBSL) is a site-specific bioconjugation platform that utilizes a Sortase (Srt) fusion protein for efficiently producing protein conjugates. Sortase is a calcium-assisted transpeptidase that is responsible for anchoring surface proteins to the peptidoglycan cell wall of Gram-positive bacteria. The enzyme cleaves the peptide bond between the amino acids T and G, within the sortase recognition motif (SRM), LPXTG. The products remain transiently attached to SrtA, until the N-terminal glycine of another protein displaces the C-terminal fragment to form a new peptide bond between the two-peptide chains.

While Srt has been used to create site-specific protein conjugates, traditionally the SRM (e.g., LPXTG) is simply inserted at the C-terminus of the protein to be labeled. Sortase is then used to link cargo-labeled peptides with an N-terminal glycine to the protein. Unfortunately, this approach is inefficient, requiring Srt to be added in excess and at high concentrations- and even then labeling is often incomplete. Proximity-Based Sortase-mediated Ligation (PBSL) overcomes this by adding a pair of low-molecular weight binding partners to both the protein and Srt, respectively (FIG. 1). These binding partners force the sortase and SRM into close proximity, increasing the efficiency of the ligation reaction to >95%. Successful ligation with cargo-labeled peptide with an N-terminal glycine, results in cleavage of the binding partners and sortase from the protein being labeled, leaving only the cargo-labeled peptide attached to the protein.

We have shown that SpyTag and SpyCacther can be used as binding partners in a PBSL system (US2020/0277403, which is incorporated by reference herein in its entirety). SpyCatcher and SpyTag are split protein-fragments from the fibronectin-binding protein that rapidly dimerize and form a covalent intermolecular amide bond in minutes amidst diverse conditions of pH, temperature and buffer. The covalent linkage prevents dissociation of SpyTag-SpyCatcher during the PBSL reaction.

There exists a further need for additional techniques to purify and conjugate proteins.

In cases where a recombinant protein does not need to be labeled with cargo, affinity tags can be used to purify the protein. The affinity tag is simply attached or fused at the N- or C-terminus of the expressed protein. Affinity tags are typically classified as either an epitope tag or a protein/domain tag. Epitope tags are usually small peptides that exhibit a high affinity towards a chromatography resin. Protein/Domain tags are used similarly, but are generally much larger. They can sometimes act as solubilizing agents, although because of their size, they can also sometimes interfere with protein fold or expression.

An advantage of an using an epitope tag for purification is that its small size will usually

not interfere with protein expression and folding. Many epitope tags are captured on chromatography resin via anti-epitope tag antibodies or large proteins (e.g., streptavidin); however, smaller capture proteins could have significant advantages such as the ability to be loaded on the resin at a much higher density. This could allow the capture of more epitope-tagged proteins. Moreover, it would be more cost-effective to generate resin with smaller capture proteins if they could be easily produced in larger quantities.

Therefore, it would be beneficial to develop new affinity purification systems based on small epitope tags and small capture proteins.

The captured epitope-labeled proteins can be released using eluting agents, low pH, denaturing conditions, or via the insertion of a protease cleavage site between the protein and epitope tag.

In addition to affinity purification, epitope tags can also be used for protein and antibody detection. Specifically, proteins or antibodies labeled with epitope tags can be detected by their binding partner or anti-epitope antibody. For detection, the binding partner (or anti-epitope antibody) can be labeled with an imaging agent or attached to a reporter enzyme.

### SUMMARY OF THE INVENTION

Described herein is the use of leader (precursor) peptides and B1 proteins, from lasso peptide biosynthesis systems, as an alternative, non-covalent option for binding partners in Proximity-Based Sortase-mediated Ligation (PBSL), affinity purification, or protein detection. Leader peptides and B1 proteins are found in actinobacteria and furmicutes. Example leader peptides include TfuA, LarA, StmA, PsmA, and PadeA; example B1 proteins include TfuB1, LarE, StmE, PsmB1, and PadeB1 (Table 1). The B1 protein recognizes the leader sequence with high affinity and specificity. Moreover, the leader peptide and B1 protein are small and easily expressed. The leader peptide can be placed at the N- or C-terminus of a desired protein and used for affinity purification, where the leader peptide is captured by the B1 protein immobilized on an affinity column/filter/beads/resin. Moreover, when the leader peptide is placed at the C-terminus it can be further used to allow purification and C-terminal ligation/labeling. In this case, the protein of interest-leader peptide fusion protein is captured by a B1 protein-Sortase enzyme fusion protein, which drives PBSL. In a separate application, proteins or antibodies that are attached to a leader peptide can be detected with a B1 protein that is labeled with an imaging agent or reporter enzyme.

According to one aspect, provided herein are conjugate protein compositions comprising a first fusion protein and a second fusion protein, wherein: (i) the first fusion protein comprises a protein of interest in series with a sortase recognition motif and a first member of a binding pair; wherein: (a) the protein of interest is N-terminal and connected via a linker to the sortase recognition motif and the sortase recognition motif is N-terminal and connected via a linker to the first binding pair member; or (b) the first binding pair member is N-terminal and connected via a linker to the sortase recognition motif and the sortase recognition motif is N-terminal and connected via a linker to the protein of interest; and (ii) the second fusion protein comprises a second binding pair member in series with a sortase and optionally a first affinity tag having a selective affinity for a first affinity tag resin, wherein: (a) the second binding pair member is N-terminal and connected via a linker to the sortase; or (b) the sortase is N-terminal and connected via a linker to the second binding pair member; wherein the members of the binding pair comprise a leader peptide and a B1 protein pair from a lasso peptide biosynthesis system that can form a heterodimer. In some embodiments, the first affinity tag is present and is connected via a linker either N-terminal or C-terminal to the second binding pair member in series with said sortase. In other embodiments, the second fusion protein does not possess an affinity tag and is chemically conjugated to a resin.

According to another aspect, provided herein are vectors encoding the proteins (e.g., the first and second fusion proteins) described herein and cells for expressing the same.

According to still another aspect, provided herein are conjugation methods, the methods comprising: (a) providing the first and the second fusion proteins from a protein conjugate composition according to embodiments described herein; (b) contacting the first fusion protein with the second fusion protein under conditions where the leader peptide forms a heterodimer with the B1 protein; and (c) adding calcium and glycine or a peptide or protein with an N-terminal glycine, under conditions where the sortase catalyzes conjugation and release of the protein of interest conjugated to the glycine or the peptide or protein with an N-terminal glycine.

According to yet another aspect, provided herein are methods for purifying a protein of interest, the methods comprising: (a) providing the first and the second fusion proteins from a protein conjugate composition according to embodiments described herein; (b) contacting the first fusion protein with the second fusion protein under conditions where the leader peptide forms a heterodimer with the B1 protein; (c) adding calcium alone or calcium and glycine, or calcium and a peptide or protein with an N-terminal glycine, under conditions where the sortase catalyzes respectively, (i) release of the protein of interest or (ii) conjugation and release of the protein of interest conjugated to the glycine or the peptide or protein with an N-terminal glycine; and (d) separating the protein of interest or the conjugated protein of interest.

According to another aspect, provided herein are conjugate protein compositions comprising a first fusion protein and a second fusion protein, wherein: (i) the first fusion protein comprises a protein of interest in series with and connected via a linker to a first binding pair member; and (ii) the second fusion protein comprises a second binding pair member optionally in series with and connected via a linker to a first affinity tag having a selective affinity for a first affinity tag resin; wherein the members of the binding pair comprise a leader peptide and a Bl protein pair from a lasso peptide biosynthesis system that can form a heterodimer. In some embodiments, the second fusion protein does not possess an affinity tag and is chemically conjugated to a resin.

According to another aspect, the first fusion protein comprises a protein of interest in series with a protease cleavage site and a first binding pair member; wherein: (a) the protein of interest is N-terminal and connected to a protease cleavages site and the protease cleavage site is N-terminal and connected via a linker to the first binding pair member; or (b) the first binding pair member is N-terminal and connected via a linker to a protease cleavage site and the protease cleavage site is connected N-terminal to the protein of interest.

According to another aspect, provided herein are vectors encoding the proteins (e.g., the first and second fusion proteins) described herein and cells for expressing the same.

According to another aspect, provided herein the second fusion protein does not possess an affinity tag and the fusion protein is chemically conjugated to a resin.

According to yet another aspect, provided herein are methods for purifying a protein of interest, the methods comprising: (a) providing the first and the second fusion proteins from a protein conjugate composition according to embodiments described herein; (b) contacting the first fusion protein with the second fusion protein under conditions where the leader peptide forms a heterodimer with the B1 protein; (c) adding an eluting agent, acidic buffer, denaturing buffer, or protease to release of the protein of interest; and (d) isolating the protein of interest.

According to yet another aspect, provided herein are methods for detecting a protein or antibody of interest, the methods comprising: (a) providing a protein or antibody connected via a linker to a first binding pair member; wherein the binding pair members comprise a leader peptide and B1 protein pair from a lasso peptide biosynthesis system that can form a heterodimer; (b) contacting the first member of binding pair member with the second binding pair member; wherein the second binding pair member is further labeled with an imaging agent or molecular reporter; and (c) detecting said imaging agent or molecular reporter.

Other features and advantages of this invention will become apparent from the following detailed description examples and figures. It should be understood, however, that the detailed description and the specific examples while indicating preferred embodiments are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application with color drawings will be provided by the Office upon request and payment of the necessary fee.

FIG. 1: Schematics of Proximity-based Sortase Ligation (PBSL) on a Solid Support.

FIG. 2. TfuA leader-TfuB1 peptide-protein pair based PBSL design.

FIGS. 3A-3B. Target protein purification through TfuB1-TfuA leader protein-peptide pair based PBSL. (FIG. 3A) Flow diagram describing purification of target proteins via PBSL using lasso leader peptide/protein binding pairs. (FIG. 3B) 4-12% SDS-PAGE showed the purification of target protein (EGFP) using TfuB1-TfuA leader protein-peptide paired PBSL.

FIGS. 4A-4B. Target protein purification+labeling through TfuB1-TfuA leader protein-peptide pair based PBSL. (FIG. 4A) Flow diagram describing purification and labeling of target proteins via PBSL) using lasso leader peptide/protein binding pairs. (FIG. 4B) 4-12% SDS-PAGE showed the purification and labeling of target protein (EGFP) with TAMRA dye using TfuB1-TfuA leader protein-peptide paired PBSL.

FIG. 5. Comparison of the target protein purification efficiency via PBSL using either an N-terminal or C-terminal fusion to a TfuA leader peptide.

FIG. 6. Comparison of protein purification efficiency via PBSL using either Lasso leader peptide/protein pairs (non-covalent) or SpyCatcher/SpyTag protein pairs (covalent).

FIG. 7. Comparison of TfuA leader-TfuB1 based PBSL to other purification systems.

FIG. 8. Schematic of affinity purification wherein the members of the binding pair comprise a leader peptide and B1 protein pair from a lasso peptide biosynthesis system.

FIG. 9. Schematic of fusion proteins (intracellular or extracellular) composed of a target protein and the first binding pair member. The cell is stained with a second binding pair member that is attached to an imaging agent or a molecular reporter. The binding pair comprise a leader peptide and B1 protein pair from a lasso peptide biosynthesis system.

### DETAILED DESCRIPTION OF THE INVENTION

The invention relates to a proximity-based sortase-mediated protein purification and ligation. Specifically, the invention relates to nucleic acid and protein conjugates and proximity-based techniques that link protein expression/purification with conjugation to therapeutic agents, imaging agents, or linkers.

Methods have been developed to link a ligand or protein of interest to its cargo, based on maleimide, N-hydroxysuccinimide, carbodiimide, and click chemistries. However, many of these suffer from poor reaction efficiencies and all of them label at random residues on the ligand. A few techniques have been developed to address these problems, including expressed protein ligation (EPL), but they have their own shortcomings.

Examples for obtaining antibody conjugates, including bispecific antibody conjugates, can be found in WO2016/183387, filed May 12, 2016, which is incorporated by reference herein in its entirety. This approach uses an antibody-binding domain (AbBD) with a photoreactive amino acid being operably linked to cargo or an antibody or a fragment thereof. Where the AbBD is fused in frame with a peptide tag or a protein that is a binding pair member and a second construct is provided comprising a second antibody or a fragment thereof and a peptide tag or a protein that is the corresponding binding pair member, the site-specific linkage of the binding pair moieties on the two constructs can be used to form a bispecific antibody. However, it is desirable to apply improved directed conjugation methods to antibodies and fragments thereof, such as antigen-binding domains or tags.

Combining the concepts behind expressed protein ligation (EPL) with a sortase enzyme, a single-step/single-construct sortase-tag expressed protein ligation (STEPL) technique has also been developed linking protein expression/purification with conjugation to therapeutic agents, imaging agents, or linkers that can be used for subsequent conjugations (e.g., biotin or click chemistry groups, such as azides or alkynes). Specifically, a coding sequence of a desired protein (e.g., a targeting ligand) was cloned in series with a coding sequence of a sortase recognition motif (e.g., LPXTG) followed by Sortase A and an affinity tag (e.g., Histidine Tag), as described in U.S. Pat. No. 9,631,218, which is incorporated by reference herein in its entirety.

However, it has unexpectedly been found that a two-construct, proximity-based sortase-mediated protein purification and ligation method yields improved results over the single-step/single-construct method in which sortase is fused directly to a desired protein. Although the latter method works well with shorter proteins, in some instances, the sortase interferes with proper folding of larger or more complex proteins (e.g., scFv proteins), thereby disrupting the secondary structure of the desired protein, and additionally, this approach may be incompatible with protein expression systems where calcium is present (e.g., yeast and mammalian systems).

The methods herein use the affinity of members of a binding pair comprising a leader peptide and a B1 protein pair from a lasso peptide biosynthesis system to achieve capture of a protein of interest, followed by subsequent cleavage of the protein and ligation of the sortase recognition motif onto the protein. By starting with the sortase and the protein of interest on separate fusion protein constructs, the secondary structure folding of the protein of interest can be achieved prior to interaction with the sortase on the capture fusion protein construct. In addition, in expression systems in which calcium is present, the two constructs can be maintained separately until the time when the interaction is set to take place.

Accordingly, in one aspect, provided herein are conjugate protein compositions comprising a first fusion protein and a second fusion protein, wherein: (i) the first fusion protein comprises a protein of interest in series with a sortase recognition motif and a first binding pair member; wherein: (a) the protein of interest is N-terminal and connected via a linker to the sortase recognition motif and the sortase recognition motif is N-terminal and connected via a linker to the first binding pair member; or (b) the first binding pair member is N-terminal and connected via a linker to the sortase recognition motif and the sortase recognition motif is N-terminal and connected via a linker to the protein of interest; and (ii) the second fusion protein comprises a second binding pair member in series with a sortase and a first affinity tag having a selective affinity for a first affinity tag resin, wherein: (a) the second binding pair member is N-terminal and connected via a linker to the sortase and the sortase is N-terminal and connected via a linker to the first affinity tag; or (b) the sortase is N-terminal and connected via a linker to the second binding pair member and the second binding pair member is N-terminal and connected via a linker to the first affinity tag; or (c) the first affinity tag is N-terminal and connected via a linker to the second binding pair member and the second binding pair member is N-terminal and connected via a linker to the sortase; or (d) the first affinity tag is N-terminal and connected via a linker to the sortase and the sortase is N-terminal and connected via a linker to the second binding pair member; wherein the members of the binding pair comprise a leader peptide and B1 protein pair from a lasso peptide biosynthesis system that can form a heterodimer.

In some embodiments, the first binding pair member is the leader peptide from the lasso

peptide biosynthesis system. In some embodiments, the leader peptide is located at the C-terminus of the first fusion protein.

In some embodiments, the leader peptide and B1 protein pair are selected from (i) the leader peptide TfuA and B1 protein TfuB1 from Thermobifida fusca; (ii) the leader peptide LarA and B1 protein LarE from Rhodococcus jostii K01-B0171; (iii) the leader peptide StmA and B1 protein StmE from Streptomonospora alba; (iv) the leader peptide PsmA and B1 protein PsmB1 from Bacillus pseudomycoides; and (v) the leader peptide PadeA and B1 protein PadeB1 from Paenibacillus dendritiformis C454. For example, the leader peptide and B1 protein pair are TfuA and TfuB1, respectively.

In some embodiments, the first fusion protein further comprises a second affinity tag having a selective affinity for a second affinity tag resin.

In some embodiments, the protein of interest is a recombinant protein, a fusion protein, and enzyme, and/or a bispecific antibody.

In some embodiments said conjugate protein composition is specifically attached in a suitable orientation to a surface, polypeptide, a particle, or a drug. In some embodiments, said additional polypeptide is a drug or a toxin.

In some embodiments, the sortase is selected from the group consisting of sortase A (SrtA), sortase B (SrtB), sortase C (SrtC), sortase D (SrtD), sortase E (SrtE) and sortase F (SrtF), and variants thereof. In one embodiment, the sortase is from Gram-positive bacteria. In one embodiment, the sortase is sortase A from Staphylococcus aureus or sortase A from Streptococcus pyogenes. In some embodiments, the sortase is engineered or modified to possess unique substrate specificity. In some embodiments, the sortase is engineered or modified to be exhibit improved or increased catalytic activity. In some embodiments, the sortase is engineered or modified to be insensitive to calcium.

In some embodiments, the sortase recognition motif is selected from the group consisting of LPXTG (SEQ ID NO: 1), LPKTG (SEQ ID NO: 2), LPATG (SEQ ID NO: 3), LPNTG (SEQ ID NO: 4), LPETG (SEQ ID NO: 5), LPXAG (SEQ ID NO: 6), LPNAG (SEQ ID NO: 7), LPXTA (SEQ ID NO: 8), LPNTA (SEQ ID NO: 9), LGXTG (SEQ ID NO: 10), LGATG (SEQ ID NO: 11), IPXTG (SEQ ID NO: 12), IPNTG (SEQ ID NO: 13), IPETG (SEQ ID NO: 14), NPQTN (SEQ ID NO: 15), LAXTG (SEQ ID NO: 16), LPXSG (SEQ ID NO: 17), LSETG (SEQ ID NO: 18), LPXCG (SEQ ID NO: 19), LPXAG (SEQ ID NO: 20), and XPETG (SEQ ID NO: 21).

In some embodiments, an affinity tag is selected from the group consisting of a histidine tag (His tag), a chitin-binding domain, a calmodulin tag, a polyglutamate tag, a maltose bidning protein, glutathione-S-transferase, an S-tag, a peptide that binds avidin/streptavidin/neutravidin (e.g. SBP-tag, Strep-tag, etc.), green fluorescent protein-tag, thioredoxin tag, Nus-tag, Fc-tag, Halo-tag. In some embodiments the affinity tag is selected from the group consisting of FLAG-tag, V5-tag, VSV-tag, Xpress tag, E-tag, Myc-tag, HA-tag, Softag, and NE-tag. In some embodiments, a protein tag may allow for specific enzymatic modification into an affinity tag, such as biotinylation by biotin ligase or BirA (e.g., AviTag, BCCP). In some embodiments, the affinity tag is selected from covalent peptide tags such as isopeptag, SpyTag, or SnoopTag. In some embodiments, the composition further comprises a first or second affinity tag resin to which said first or second affinity tag, respectively, selectively binds. In some embodiments, the resin is an immobilized metal affinity chromatography (IMAC) resin. In some embodiments, the resin is selected from the group consisting of nickel resin, cobalt resin, TALON® resin, chitin resin, and streptavidin resin. In some embodiments, said affinity tag and said resin is selected from the group of combinations consisting of a histidine tag (His tag) in combination with a nickel or cobalt resin, a chitin-binding domain affinity tag in combination with a chitin resin, and biotinylated biotin acceptor peptide affinity tag in combination with a streptavidin resin. In one embodiment, the affinity tag is a histidine tag and the resin is a nickel or a cobalt resin. In some embodiments, the resin is bound to an antibody capable of binding the affinity tag. In some embodiments, the resin is bound to a protein capable of binding the affinity tag, such as avidin/streptavidin/neutravidin, streptactin, calmodulin, Protein A or G, or S-protein. In some embodiments, the resin is HaloLink resin. In some embodiments, the resin is amylose agarose. In some embodiments glutathione is bound to the resin.

In some embodiments, the N-terminal glycine comprises a single glycine. In some embodiments, the N-terminal glycine comprises a plurality of N-terminal glycines or an N-terminal polyglycine, e.g., an N-terminal triglycine. In some embodiments, the glycine, polyglycine, or peptide/protein (including enzymes) with an N-terminal glycine further comprises a functional group or label. In some embodiments, the glycine, polyglycine, or peptide/protein with an N-terminal glycine is fused or linked to a protein, an enzyme, a drug molecule, an imaging agent, a metal chelate, a polyethylene glycol, a click chemistry group, an alkyne, an azide, a hapten, a biotin, a photocrosslinker, an oligonucleotide, a small molecule, azodibenzocyclooctyne (ADIBO), DIG, DBCO, TCO, tetrazine, a nanoparticle, or an antibody binding domain (AbBD).

In some embodiments, the peptide/protein with an N-terminal glycine is fused or linked to the protein of interest to permit circularization with the protein, to allow circularization and purification of the protein in a single step. In one embodiment, the click chemistry group comprises GGG-K (azide) or an azodibenzocyclooctyne (ADIBO)-functionalized superparamagnetic iron oxide (SPIO) nanoparticle. In one embodiment, the imaging agent comprises a fluorophore or a ligand capable of chelating a metal or radioisotope. In one embodiment, the drug molecule comprises an antibiotic. In some embodiments, the protein of interest is an antibody binding domain (AbBD) that comprises Protein A, Protein G, Protein L, CD4, or a subdomain thereof. In some embodiments, said subdomain is an engineered subdomain, such as to include a non-natural amino acid, a photoreactive group, or a crosslinker. In some embodiments, the antibody-binding domain (AbBD) is operably linked to a photoreactive amino acid and is operably linked to an antibody or a fragment thereof. In one embodiment, said antibody-binding domain (AbBD) is operably linked to an immunoglobulin Fc region, such as an IgG. In one embodiment, said photoreactive amino acid is a UV-active non-natural amino acid or benzoylphenylalaine (BPA). In some embodiments, said antibody-binding domain is a domain of Protein G, Protein A, Protein L, or CD4 or is hyperthermophilic variant of the B1 domain of protein G (HTB1). In some embodiments, BPA is incorporated into a protein Z comprising SEQ ID NO: 22, such as to replace F5, F13, L17, N23, Q32, or K35 of SEQ ID NO: 22. In some embodiments, BPA is incorporated into a protein G domain comprising SEQ ID NO: 23, such as to replace A24 or K28 of SEQ ID NO: 23.

Generally, a conjugate protein composition may be specifically attached in the proper orientation to a surface or a particle.

In one embodiment, the linkers comprise glycine-serine (GS)-rich linkers. In one embodiment, glycine-serine (GS)-rich linkers are (GGS), linkers, where n is an integer indicating the number of (GGS) repeats, such as where n is an integer greater or equal to 2 and or where n is an integer between 2 and 5, both inclusive. In some embodiments, the linker is a (GGS) 5 linker. The (GGS) s linker facilitates the sortase domain to have the conformational freedom to recognize the sortase recognition motif. Alternatively, the linker is a (GGS) 2 or (GGS) 3 linker. Notably, five GGS repeats may be chosen for the fusion construct because the crystal structure reports a length of 26.2 Å between the N-terminus of the sortase domain and its active site, corresponding to the length of approximately 3 GGS repeats (8.8 Å each). Thus, a (GGS) 5 linker may be expected to provide sufficient spatial flexibility for the sortase domain to recognize and bind the LPXTG motif.

In some embodiments, an antibody or antibody fragment comprises immunoglobulin G (IgG), immunoglobulin M (IgM), immunoglobulin D (IgD), immunoglobulin E (IgE), or immunoglobulin A (IgA). In some embodiments, the IgG is selected from the group consisting of IgG1, IgG2, IgG3, and IgG4. In some embodiments, the antibody fragment comprises an Fc domain or an Fab domain. In some embodiments, the antibody fragment comprises an Fv, Fab, Fab′, or (Fab′)2 domain. In some embodiments, the antibody fragment comprises a variable region of an antibody, a single-chain antibody, or an scFv. In some embodiments, said antibody or fragment thereof comprises an scFv-Fc or other fusion antibody.

In another aspect, provided herein are vectors encoding a first fusion protein and/or a second fusion protein described herein. In some embodiments, the vector is an expression vector.

In still another aspect, provided herein are cells for recombinantly expressing the first fusion protein and/or the second fusion protein, wherein the cell is a bacterial cell, a yeast cell, an insect cell, or a mammalian cell. In some embodiments, the cell is transformed with an expression vector described herein.

In yet another aspect, provided herein are conjugation methods, the methods comprising: (a) providing the first and the second fusion proteins from a protein conjugate composition according to embodiments described herein; (b) contacting the first fusion protein with the second fusion protein under conditions where the leader peptide forms a heterodimer with the B1 protein; and (c) adding calcium and glycine or a peptide or protein with an N-terminal glycine, under conditions where the sortase catalyzes conjugation and release of the protein of interest conjugated to the glycine or the peptide or protein with an N-terminal glycine.

According to yet another aspect, provided herein are methods for purifying a protein of interest, the methods comprising: (a) providing the first and the second fusion proteins from a protein conjugate composition according to embodiments described herein; (b) contacting the first fusion protein with the second fusion protein under conditions where the leader peptide forms a heterodimer with the B1 protein; (c) adding calcium alone or calcium and glycine, or calcium and a peptide or protein with an N-terminal glycine, under conditions where the sortase catalyzes respectively, (i) release of the protein of interest or (ii) conjugation and release of the protein of interest conjugated to the glycine or the peptide or protein with an N-terminal glycine; and (d) separating the protein of interest or the conjugated protein of interest.

According to another aspect, provided herein are conjugate protein compositions comprising a first fusion protein and a second fusion protein, wherein: (i) the first fusion protein comprises a protein of interest in series with and connected via a linker to a first binding pair member; and (ii) the second fusion protein comprises a second binding pair member optionally in series with and connected via a linker to a first affinity tag having a selective affinity for a first affinity tag resin; wherein the members of the binding pair comprise a leader peptide and a B1 protein pair from a lasso peptide biosynthesis system that can form a heterodimer. In some embodiments, the second fusion protein does not possess an affinity tag and is chemically conjugated to a resin.

According to another aspect, the first fusion protein comprises a protein of interest in series with a protease cleavage site and a first binding pair member; wherein: (a) the protein of interest is N-terminal and connected to a protease cleavages site and the protease cleavage site is N-terminal and connected via a linker to the first binding pair member; or (b) the first binding pair member is N-terminal and connected via a linker to a protease cleavage site and the protease cleavage site is connected N-terminal to the protein of interest.

According to another aspect, provided herein are vectors encoding the proteins (e.g., the first and second fusion proteins) described herein and cells for expressing the same.

According to another aspect, provided herein the second fusion protein does not possess an affinity tag and the fusion protein is chemically conjugated to a resin. According to yet another aspect, provided herein are methods for purifying a protein of interest, the methods comprising: (a) providing the first and the second fusion proteins from a protein conjugate composition according to embodiments described herein; (b) contacting the first fusion protein with the second fusion protein under conditions where the leader peptide forms a heterodimer with the B1 protein; (c) adding an eluting agent, acidic buffer, denaturing buffer, or protease to release of the protein of interest; and (d) isolating the protein of interest.

According to yet another aspect, provided herein are methods for detecting a protein or antibody of interest, the methods comprising: (a) providing a protein or antibody connected via a linker to a first binding pair member; wherein the binding pair members comprise a leader peptide and B1 protein pair from a lasso peptide biosynthesis system that can form a heterodimer; (b) contacting the first member of binding pair member with the second binding pair member; wherein the second binding pair member is further labeled with an imaging agent or molecular reporter; and (c) detecting said imaging agent or molecular reporter.

In a preferred embodiment, the sortase recognition motif includes the motif LPXTG (Leu-Pro-any-Thr-Gly—SEQ ID NO: 1) (wherein the occurrence of X represents independently any amino acid residue). Sortase cleaves between the Gly and Thr of the LPXTG motif. Other sortase recognition motifs, known in the art, can also be used. Examples of such sortase recognition motifs are described in WO2013/003555, U.S. Pat. No. 7,238,489 and US 2014/0030697, which are each incorporated by reference herein in its entirety. Examples of other sortase recognition motifs, include, but are not limited to LPKTG (SEQ ID NO: 2), LPATG (SEQ ID NO: 3), LPNTG (SEQ ID NO: 4), LPETG (SEQ ID NO: 5), LPXAG (SEQ ID NO: 6), LPNAG (SEQ ID NO: 7), LPXTA (SEQ ID NO: 8), LPNTA (SEQ ID NO: 9), LGXTG (SEQ ID NO: 10), LGATG (SEQ ID NO: 11), IPXTG (SEQ ID NO: 12), IPNTG (SEQ ID NO: 13), IPETG (SEQ ID NO: 14). Additional suitable sortase recognition motifs, such as NPQTN (SEQ ID NO: 15), will be apparent to one skilled in the art, and the invention is not limited in this respect.

The coding sequence of a suitable sortase enzyme can be used. Sortases are known in the art. Sortases are also referred to as transamidases, and typically exhibit both a protease and a transpeptidation activity. Sortases have been classified into 6 classes, designated A, B, C, D, E, and F; designated sortase A (SrtA), sortase B (SrtB), sortase C (SrtC), sortase D (SrtD), sortase E (SrtE), and sortase F (SrtF), respectively, based on sequence alignment and phylogenetic analysis of sortases from Gram-positive bacterial genomes. In a preferred embodiment, sortase is sortase A. In some embodiments, the sortase A is from Staphylococcus aureus or from Streptococcus pyogenes. The coding sequences of sortases, including sortase A, are known and publicly available in biological sequence databases and U.S. Pat. No. 7,238,489, which is incorporated by reference herein in its entirety.

Any suitable affinity tag known to one skilled in the art can be used. In one embodiment, the affinity tag is a histidine tag (His tag).

Also provided herein are vectors encoding a conjugate or fusion protein described herein. Any suitable expression vector known to one of skilled in the art can be used. The expression protocol can be optimized based on the chosen vector.

Following protein expression and capture through interaction between the binding pair partners, the protein of interest can be released from the sortase and affinity tag by administration of calcium and glycine. Peptides or proteins with one or more N-terminal glycines are possible. During this process glycine, the glycine-peptide/protein is specifically ligated to the C-terminus of the protein of interest. This method therefore allows for the facile conjugation of a peptide/protein specifically to the C-terminus of the expressed protein.

For example, using the peptide GGG-K (FAM) allows attachment of the fluorescent dye fluorescein (FAM) to the C-terminus of the expressed protein. This dye can be ligated, for example, in a 1:1 stoichiometric ratio with the expressed protein. Therefore, it is site-specific and can be used for quantitative analysis of fluorescence.

A suitable molecule that can be attached to a peptide with an N-terminal glycine can be specifically attached to the C-terminus of the expressed protein (e.g., dyes, drugs, haptens such as biotin, polymers such as PEG, etc.).

In one example, a peptide is ligated with an azide group (e.g. GGG-K (azide)), which is subsequently used for click conjugation reactions. For example, after ligation click chemistry can be used to attach the expressed protein onto surfaces (e.g., for ELISA assays and nanoparticle surfaces). Importantly, the conjugation in this case is site-specific, so the proteins are all oriented in the same direction on the surface. Moreover, there is only a single attachment point-the azide—which is ligated to the C-terminus of the protein in a 1:1 ratio. One could also click drug molecules or other agents to the expressed protein, in a site-specific manner.

Following protein expression and capture through interaction between the binding pair partners, the protein of interest can be released from the sortase and affinity tag by administration of calcium and glycine. Peptides or proteins with one or more N-terminal glycines are preferred. If the sortase recognition motif is N-terminal to the protein of interest, the released protein will possess a glycine that is N-terminal to the protein of interest.

A general vector for bacterial expression has been produced. The expression protocol has been optimized. The cleavage reaction has been studied quantitatively and modeled to allow for optimization based on the user's needs. The system has been successfully used to express and conjugate a number of proteins including eGFP (EGFP), affibodies, IgG, antibody fragments (e.g., scFv's), natural extracellular matrix binding domains, and cytokines. The conjugated peptides have included visible and near-IR fluorophores, drugs (e.g., MMAE), haptens (e.g., biotin), polymers (e.g., PEG), and bio-orthogonal reactive groups (e.g., azide).

In addition to calcium for cleavage, any suitable agent known to one skilled in the art can be used. For example, one can reengineer the sortase domain to be calcium independent or to depend on a transition metal or small molecule rather than calcium for cleavage.

The purification or conjugation systems described here have advantages over expressed protein ligation and other sortase-mediated purification or conjugation systems. First, the techniques here link the final purification step to conjugation, ensuring that recovered protein is conjugated. This eliminates the difficult separation of conjugated and unconjugated peptides or proteins. Second, placing the protein of interest N-terminal to the LPXTG motif allows the first, glycine-free step in the sortase mechanism to occur without releasing any protein. Because the sortase retains the protein during this step, the crippling W194A mutation (required in other sortase purification techniques) is unnecessary and the more efficient wild-type Sa-SrtA can be used. The system also avoids chemistry based on functional groups generally found in biology, such as amines and thiols, greatly expanding the classes of proteins that can be expressed.

The methods and compositions described herein can be used in recombinant protein expression and other applications. For example, these applications include efficiently and economically producing targeting ligands conjugated to imaging and therapeutic agents. Another is PEGylation of a biologic drug to help improve its circulation time. An additional use is the ligation of unique chemical moieties (e.g., click groups such as azides or alkynes, biotin, DIG, etc), at the C-terminus of the expressed protein to allow facile and site-specific conjugation to surfaces, drugs, imaging agents, nanoparticles, etc. Applications also include protein purification. Proteins can be produced with extremely high purity levels because the sortase reaction triggers the release of only the protein of interest. Other proteins that are non-specifically bound to the affinity column are not released upon the addition of glycine and/or calcium. Moreover, affinity tags with superior affinity (e.g., His12 vs. His6) can used, because protein purification does not require stripping the protein of interest from the affinity column. Rather, the protein is released via the sortase reaction. This is important because it allows the protein to be subjected to more stringent washing conditions when bound to the affinity column, prior to sortase-mediated release. This is not possible with other systems because when affinity tags are too tightly bound to the affinity column, the harsh conditions that are necessary to eventually release the protein from the affinity column can be damaging to that protein.

In addition, the techniques described here can be used to functionalize targeting ligands with chemical groups useful for molecular imaging. For example, a ligand can be used to chelate metals (e.g., Gd) or radioisotopes (e.g., Cu-64) for magnetic resonance, CT, or nuclear imaging. As another example, a near-IR fluorophore can be used to optically differentiate between cells expressing and lacking a proto-oncogene, such as Her2/neu. In one example, an NIR-dyed affibody is used to quantify Her2/neu expression differences between different cells (e.g., T6-17 cells, NIH/3T3 cells, cancerous or non-cancerous cells from patient samples), which demonstrates its utility for in-cell Western techniques. Additionally, proximity-based sortase-mediated expressed protein ligation can be used to conjugate a bio-orthogonal reactive group (e.g., an azide to the Her2/neu affibody of this example). For example, azide's ability to react to a strained alkyne present on the surface of superparamagnetic iron oxide nanoparticles can be observed. Due to the site-specificity of proximity-based sortase-mediated expressed protein ligation, the affibody can be linked in a specific orientation, which would increase the particle's efficacy in distinguishing between cells expressing and lacking Her2/neu. Proximity-based sortase-mediated expressed protein ligation can also be used to conjugate many other moieties to a target protein, such as biotin, poly(ethylene-glycol), antibiotics, metal chelates, and photo-crosslinkers, all of which have been proven compatible with the sortase enzyme.

In one embodiment, the protocol is modified, optimized, modeled, and used to conjugate a Her2/neu or EGFR-targeting affibody to a fluorophores for imaging and/or to an azide for subsequent copper-free click chemistry reactions with azadibenzocyclooctyne (ADIBO)-functionalized superparamagnetic iron oxide nanoparticles, demonstrating the system's flexibility, efficacy, and utility.

Provided herein are protein or antibody conjugates (e.g., a bispecific antibody), drug and nanoparticle compositions, as well as methods and compositions to generate them. Further provided herein are methods of using the compositions to image, diagnose or treat a disease, such as cancer.

All types of antibodies are contemplated. Described herein are methods to site-specifically label an antibody with a chemical or biological moiety. Provided herein are also methods to site-specifically attach an antibody onto a surface. Also provided herein methods of producing a bispecific antibody. The inventors have developed facile methods to efficiently produce bispecific antibodies from full-length IgG, by ligating a second targeting ligand with an N-terminal glycine. More broadly, the inventors have developed facile methods to efficiently produce bispecific targeting ligands with a protein of interest being the first targeting ligand, which can then be ligated to a second targeting ligand with an N-terminal glycine.

“Protein Z” refers to a Z domain based on the B domain of Staphylococcal aureus Protein A. The wild-type Protein Z amino acid sequence is: VDNKFNKEQQNAFYEILHLPNLNEEQRNAFIQSLKDDPSQSANLLAEAKKLNDAQAPK MRM (SEQ ID NO: 22). Photoreactive Protein Z includes those where an amino acid in protein Z has been replaced with benzoylphenylalanine (BPA), such as F13BPA and F5BPA (underlined amino acids in bold in SEQ ID NO: 22). Examples of other BPA-containing Protein Z mutants include, but are not limited to, Q32BPA, K35BPA, N28BPA, N23BPA, and L17BPA. Examples of Protein Z variants or mutants include, F5I, such as F5I K35BPA. The Protein Z amino acid sequence may also include homologous, variant, and fragment sequences having Z domain function. In some embodiments, the Protein Z amino acid sequence includes an amino acid sequence which is 60, 65, 70, 75, 80, 85, 90, 95, or 99% identity to the sequence set forth in SEQ ID NO: 22.

“Protein G” refers to a B1 domain based on Streptococcal Protein G. Preferably, the Protein G is a hyperthermophilic variant of a B1 domain based on Streptococcal Protein G. The Protein G amino acid sequence preferably is: MTFKLIINGKTLKGEITIEAVDAAEAEKIFKQYANDYGIDGEWTYDDATKTFTVTE (SEQ ID NO: 23). Nine Protein G variants were successfully designed and expressed, each with an Fc-facing amino acid substituted by BPA: V21, A24, K28, 129, K31, Q32, D40, E42, W42(underlined and bold in SEQ ID NO: 23). Two variants, A24BPA and K28BPA, allowed ˜100% of all human IgG subtypes to be labeled. The Protein G amino acid sequence may also include homologous, variant, and fragment sequences having B1 domain function. In some embodiments, the Protein G amino acid sequence includes an amino acid sequence which is 60, 65, 70, 75, 80, 85, 90, 95, or 99% identity to the sequence set forth in SEQ ID NO: 23.

The term “Fc domain” encompasses the constant region of an immunoglobulin molecule. The Fc region of an antibody interacts with various Fc receptors and ligands, imparting an array of important functional capabilities referred to as effector functions. For IgG, the Fc region comprises Ig domains CH2 and CH3. An important family of Fc receptors for the IgG isotype are Fc gamma receptors (FcγRs). These receptors mediate communication between antibodies and the cellular arm of the immune system.

A “Fab domain” encompasses an antibody region that binds to antigens. The Fab region is composed of one constant and one variable domain of each of the heavy and light chains.

The term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. The term “modified immunoglobulin G” refers to a molecule derived from an antibody of the “G” class. The term “antibody” refers to a protein of one or more polypeptides substantially encoded by all or part of a recognized immunoglobulin gene. The recognized immunoglobulin genes, for example in humans, include the kappa (κ), lambda (λ) and heavy chain genetic loci, which together comprise the myriad variable region genes, and the constant region genes mu (μ) delta (δ), gamma (γ), sigma (σ) and alpha (α) which encode the IgM, IgD, IgG, IgE, and IgA isotypes or classes, respectively. An “antibody” is meant to include full-length antibodies, and may refer to a natural antibody from any organism, an engineered antibody, or an antibody generated recombinantly for experimental, therapeutic, or other purposes. Furthermore, full-length antibodies comprise conjugates as described and exemplified herein. The term “antibody” encompasses monoclonal and polyclonal antibodies. Antibodies can be antagonists, agonists, neutralizing, inhibitory, or stimulatory. Specifically included as an “antibody” are full-length antibodies described and exemplified herein. By “full length antibody” herein is meant the structure that constitutes the natural biological form of an antibody, including variable and constant regions.

An antibody “variable region” contains the antigen binding determinants of the molecule, and thus determines the specificity of an antibody for its target antigen. The variable region is so named because it is the most distinct in sequence from other antibodies within the same isotype. The majority of sequence variability occurs in complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e., bi-specific) hybrid antibodies (e.g., Lanzavecchia et al., Eur. J. Immunol. 17:105 (1987)) and in single chains (e.g., Huston et al., Proc. Natl. Acad. Sci. 85:5879-5883 (1988) and Bird et al., Science (1988) 242:423-426, which are incorporated herein by reference). (See, generally, Hood et al., “Immunology”, Benjamin, N.Y., 2nd ed. (1984), and Hunkapiller & Hood, Nature (1986) 323:15-16).

An “epitope” refers to a region of an antigen that binds to the antibody or antigen-binding fragment. It is the antigen region recognized by a first antibody where the binding of the first antibody to the region prevents binding of a second antibody or other bivalent molecule to the region. The region encompasses a particular core sequence or sequences selectively recognized by a class of antibodies. In general, epitopes are comprised of local surface structures that can be formed by contiguous or noncontiguous amino acid sequences.

The terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art, such as, for example, ELISA or cold displacement assays.

As “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, Cγ1, Cγ2, and Cγ3. In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms including full-length antibodies, antibody fragments, and individual immunoglobulin domains including VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

The term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.
(6) scFv-Fc, is produced in one embodiment, by fusing single-chain Fv (scFv) with a hinge region from an immunoglobulin (Ig) such as an IgG, and Fc regions.

In some embodiments, the antibody is a monoclonal antibody. In some embodiments, the antigen-binding fragment is a single chain Fv (scFv), a diabody, a tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

The terms “bivalent molecule” or “BV” refer to a molecule capable of binding to two separate targets at the same time. A bivalent molecule is not limited to having two and only two binding domains and can be a polyvalent molecule or a molecule comprised of linked monovalent molecules. The binding domains of a bivalent molecule can selectively recognize the same epitope or different epitopes located on the same target or located on a target that originates from different species. The binding domains can be linked in any of a number of ways including disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983) (general review); Morley, J. S., Trends Pharm Sci (1980) pp. 463-468 (general review); Hudson et al., Int J Pept Prot Res (1979) 14:177-185; Spatola et al., Life Sci (1986) 38:1243-1249; Hann, M., J Chem Soc Perkin Trans I (1982) 307-314; Almquist et al., J Med Chem (1980) 23:1392-1398; Jennings-White et al., Tetrahedron Lett (1982) 23:2533; Szelke et al., EP 45,665; Chemical Abstracts 97, 39405 (1982); Holladay et al., Tetrahedron Lett (1983) 24:4401-4404; and Hruby, V. J., Life Sci (1982) 31:189-199).

The terms “binds” or “binding” or grammatical equivalents, refer to compositions having affinity for each other. “Specific binding” is where the binding is selective between two molecules. A particular example of specific binding is that which occurs between an antibody and antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

A “dimer” is a macromolecular complex formed by two macromolecules, usually proteins (or portions thereof) or nucleic acids (or portions thereof). A “homodimer” is formed by two identical macromolecules (“homodimerization”), while a “heterodimer” is formed by two distinct macromolecules (“heterodimerization”). Many dimers are non-covalently linked, but some (e.g., NEMO homodimers) can link via, e.g., disulfide bonds. Some proteins have regions specialized for dimerization, known as “dimerization domains.” In some cases, a truncated protein containing a dimerization domain (or two truncated proteins containing corresponding dimerization domains) may be able to interact in the absence of one or both complete protein sequence(s). Similarly, a fusion protein including a dimerization domain (or two fusion proteins including corresponding dimerization domains) may be able to interact in the absence of one or both complete protein sequence(s). Mutations to these domains may increase, or alternatively reduce, dimer formation.

In one embodiment, an antibody or antigen-binding fragment binds its target with a K_{D} of 0.1-10 mM. In one embodiment, an antibody or antigen-binding fragment binds its target with a K_{D} of 0.1-1 mM. In one embodiment, an antibody or antigen-binding fragment binds its target with a K_{D} within the 0.1 nM range. In one embodiment, an antibody or antigen-binding fragment binds its target with a K_{D} of 0.1-2 nM. In one embodiment, an antibody or antigen-binding fragment binds its target with a K_{D} of 0.1-1 nM. In oner embodiment, an antibody or antigen-binding fragment binds its target with a K_{D} of 0.05-1 nM. In one embodiment, an antibody or antigen-binding fragment binds its target with a K_{D} of 0.1-0.5 nM. In one embodiment, an antibody or antigen-binding fragment binds its target with a K_{D} of 0.1-0.2 nM.

In some embodiments, the antibody or antigen-binding fragment thereof comprises a modification. In some embodiments, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody or other cellular ligand, etc. Any of numerous chemical modifications may be carried out by known techniques including specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

In some embodiments, the modification is an N-terminus modification. In some embodiments, the modification is a C-terminal modification. In some embodiments, the modification is N-terminal biotinylation. In some embodiments, the modification is C-terminal biotinylation. In some embodiments, the secretable form of the antibody or antigen-binding fragment has an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment has an N-terminal modification that allows binding to an enzymatically biotinylatable site. In some embodiments, the secretable form of the antibody or antigen-binding fragment has a C-terminal modification that allows binding to an enzymatically biotinylatable site. In some embodiments, biotinylation of the site functionalizes the site to bind to a surface coated with streptavidin, avidin, avidin-derived moieties, or a secondary reagent.

It will be appreciated that a “modification” can encompass an amino acid modification such as an amino acid substitution, insertion, and/or deletion in a polypeptide sequence.

A variety of radioactive isotopes are available to produce radio-conjugate antibodies and other proteins that can be used in the methods and compositions described here. Examples include At211, Cu64, I131, I125, Y90, Re186, Re188, Sm153, Bi212, P32, Zr89 and radioactive isotopes of Lu.

In other embodiments, enzymatically active toxin or fragments thereof that can be used include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

A chemotherapeutic or other cytotoxic agent may be conjugated to the protein, according to methods described herein, as an active drug or as a prodrug. A “prodrug” refers to a precursor or derivative form of a pharmaceutically active substance that is less cytotoxic to tumor cells compared to the parent drug and is capable of being enzymatically activated or converted into the more active parent form. Prodrugs that may be used include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug.

In one embodiment, a combination of the protein with the biological active agents specified above, i.e., a cytokine, an enzyme, a chemokine, a radioisotope, an enzymatically active toxin, or a chemotherapeutic agent can be applied.

A variety of other therapeutic agents may find use for administration with the antibodies and conjugates described herein. In one embodiment, the conjugate is administered with an anti-angiogenic agent. An “anti-angiogenic agent” refers to a compound that blocks, or interferes to some degree, with blood vessel development. It may, for instance, be a small molecule or a protein, for example an antibody, Fc fusion, or cytokine, that binds to a growth factor or growth factor receptor involved in promoting angiogenesis. In another embodiment, the conjugate is administered with a therapeutic agent that induces or enhances an adaptive immune response. In another embodiment, the conjugate is administered with a tyrosine kinase inhibitor. A “tyrosine kinase inhibitor” refers to a molecule that inhibits to some extent tyrosine kinase activity of a tyrosine kinase.

In one embodiment, conjugates described herein may be used for various therapeutic purposes. In one embodiment, the conjugates are administered to a subject to treat an antibody-related disorder. In another embodiment, the conjugates are administered to a subject to treat a tumor or a cancer tumor. A “subject” for the purposes described herein includes humans and other animals, preferably mammals and most preferably humans. The subject may include dogs, cats, pigs, cows, sheep, goats, horses, rats, and mice and humans. The term “subject” does not exclude an individual that is normal in all respects.

Thus, the conjugates provided herein have both human therapy and veterinary applications. In one embodiment the subject is a mammal, and in one embodiment the mammal is a human. A “condition” or “disease” includes a disorder that may be ameliorated by the administration of a pharmaceutical composition comprising a conjugate or by a method provided herein. Antibody related disorders include, but are not limited to, autoimmune diseases, immunological diseases, infectious diseases, inflammatory diseases, neurological diseases, and oncological and neoplastic diseases including cancer.

Provided herein are nucleic acid constructs encoding the conjugates and fusion proteins herein. A “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. The term includes oligonucleotides composed of naturally occurring nucleobases, sugars and covalent inter-nucleoside (backbone) linkages, as well as oligonucleotides having non-naturally-occurring portions, which function similarly. Such modified or substituted oligonucleotides are often preferred over native forms because of desirable properties, such as enhanced cellular uptake, enhanced affinity for nucleic acid target and increased stability in the presence of nucleases.

In some embodiments, the vector comprises a nucleic acid encoding a protein, polypeptide, peptide, antibody, or recombinant protein described herein. In one embodiment, provided herein are vectors comprising nucleic acids encoding fusion proteins described herein.

In one embodiment, the nucleic acid can be expressed in a variety of different systems, in vitro and in vivo, according to the desired purpose. For example, a nucleic acid can be inserted into an expression vector, introduced into a desired host, and cultured under conditions effective to achieve expression of a polypeptide encoded by the nucleic acid. Effective conditions include culture conditions which are suitable for achieving production of the polypeptide by the host cell, including effective temperatures, pH, media, additives to the media in which the host cell is cultured (e.g., additives which amplify or induce expression such as butyrate, or methotrexate if the coding nucleic acid is adjacent to a dhfr gene), cycloheximide, cell densities, culture dishes, etc. In another embodiment, a nucleic acid is introduced into the cell by an effective method including, e.g., naked DNA, calcium phosphate precipitation, electroporation, injection, DEAE-Dextran mediated transfection, fusion with liposomes, association with agents which enhance its uptake into cells, viral transfection. A cell into which the nucleic acid has been introduced is a transformed host cell. The nucleic acid can be extrachromosomal or integrated into a chromosome(s) of the host cell. It can be stable or transient. An expression vector is selected for its compatibility with the host cell. Host cells include, mammalian cells (e.g., COS-7, CV1, BHK, CHO, HeLa, LTK, NIH 3T3, 293, PAE, human, human fibroblast, human primary tumor cells, testes cells), insect cells, such as Sf9 (S. frugipeda) and Drosophila, bacteria, such as E. coli, Streptococcus, bacillus, yeast, such as S. cerevisiae (e.g., cdc mutants, cdc25, cell cycle and division mutants, such as ATCC Nos. 42563, 46572, 46573, 44822, 44823, 46590, 46605, 42414, 44824, 42029, 44825, 44826, 42413, 200626, 28199, 200238, 74155, 44827, 74154, 74099, 201204, 48894, 42564, 201487, 48893, 28199, 38598, 201391, 201392), fungal cells, plant cells, embryonic stem cells (e.g., mammalian, such as mouse or human), fibroblasts, muscle cells, neuronal cells, etc. Expression control sequences are similarly selected for host compatibility and a desired purpose, e.g., high copy number, high amounts, induction, amplification, controlled expression. Other sequences that can be used include enhancers such as from SV40, CMV, RSV, inducible promoters, cell-type specific elements, or sequences which allow selective or specific cell expression. Promoters that can be used to drive expression, include an endogenous promoter, promoters of other genes in a cell signal transduction pathway, MMTV, SV40, trp, lac, tac, or T7 promoters for bacterial hosts; or alpha factor, alcohol oxidase, or PGH promoters for yeast.

In one embodiment, reporter genes are incorporated within expression constructs to facilitate identification of transcribed products. Accordingly, in one embodiment, reporter genes used are selected from the group consisting of β-galactosidase, chloramphenicol acetyl transferase, luciferase and a fluorescent protein.

In one embodiment, the conjugates are purified or isolated after expression. Proteins may be isolated or purified in a variety of ways known to those skilled in the art. Purification methods include chromatographic techniques, including ion exchange, hydrophobic interaction, affinity, sizing or gel filtration, and reversed-phase, carried out at atmospheric pressure or at high pressure using systems such as FPLC or HPLC. Purification methods also include electrophoretic, immunological, precipitation, dialysis, and chromatofocusing techniques. Ultrafiltration and diafiltration techniques, in conjunction with protein concentration, are also useful. As is known in the art, a variety of natural proteins bind Fc and antibodies, and these proteins can find use for purification of conjugates described herein. For example, the bacterial proteins A and G bind to the Fc region. Likewise, the bacterial protein L binds to the Fab region of some antibodies, as of course does the antibody's target antigen. Purification can often be enabled by a particular fusion partner. For example, proteins may be purified using glutathione resin if a GST fusion is employed, Ni⁺² affinity chromatography if a His-tag is employed, or immobilized anti-flag antibody if a flag-tag is used. The degree of purification needed will vary depending on the screen or use of the conjugates. In some instances, no purification is necessary. For example, if conjugates are secreted, screening may take place directly from the media. As known in the art, some selection methods do not involve purification of proteins. For example, if conjugates are made into a phage display library, protein purification may not be performed.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

There are many options for linking modules. A variety of linkers may be used to generate conjugates and fusion proteins. The term “linker,” “linker sequence,” “spacer,” “tethering sequence” or grammatical equivalents thereof refer to a molecule or group of molecules (such as a monomer or polymer) that connects two molecules and often serves to place the two in a preferred configuration. Several strategies may be used to covalently link molecules together. These include, but are not limited to, polypeptide linkages between N- and C-termini of proteins or protein domains, linkage via disulfide bonds, and linkage via chemical cross-linking reagents. In one embodiment, the linker is a peptide bond, generated by recombinant techniques or peptide synthesis. In another embodiment, the linker is a cysteine linker. In yet another embodiment, it is a multi-cysteine linker. Choosing a suitable linker for a specific case where two polypeptide chains are to be connected depends on various parameters including, but not limited to, the nature of the two polypeptide chains (e.g., whether they naturally oligomerize) and the distance between the N- and C-termini to be connected, if known, and/or the stability of the linker towards proteolysis and oxidation. Furthermore, a linker may contain amino acid residues to provide flexibility. Thus, the linker peptide may predominantly include the amino acid residues: Gly, Ser, Ala, and Thr. The linker peptide should be adequately long to link two molecules in such a way that they assume the correct conformation relative to one another to retain the desired activity. Suitable lengths include at least one and not more than 30 amino acid residues. In one embodiment, the linker is from 1 to 30 amino acids long. In another embodiment, the linker is from 1 to 15 amino acids long. In addition, the amino acid residues selected should have properties that do not significantly interfere with the polypeptide's activity. Thus, the linker peptide overall should not have a charge inconsistent with the polypeptide's activity, or interfere with internal folding, or form bonds or other interactions with amino acid residues in one or more of the monomers that would seriously impede the binding of receptor monomer domains. Useful linkers include glycine-serine polymers, glycine-alanine polymers, alanine-serine polymers, and other flexible linkers such as the tether for the shaker potassium channel, and a large variety of other flexible linkers, as will be appreciated by those in the art. Suitable linkers may also be identified by screening databases of known three-dimensional structures for naturally occurring motifs that can bridge the gap between two polypeptide chains. In one embodiment, the linker is not immunogenic when administered in a human. Thus, linkers may be chosen such that they have or are thought to have low immunogenicity. Another way of obtaining a suitable linker is to optimize a simple linker, e.g., (Gly₄Ser)ₙ, through random mutagenesis. Alternatively, once a suitable polypeptide linker is defined, additional linker polypeptides can be created to select amino acids that more optimally interact with the domains being linked. Other types of linkers that may be used include artificial polypeptide linkers and inteins. In another embodiment, disulfide bonds are designed to link two molecules. In another embodiment, linkers are chemical cross-linking agents. For example, a variety of bifunctional protein coupling agents may be used, such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In another embodiment, chemical linkers may allow chelation of an isotope. Carbon-14-labeled 1-isothiocyanatobenzyl-3-methyldiethylene triaminepentaacetic acid (MX-DTPA) is an exemplary chelating agent to conjugate a radionucleotide to an antibody. The linker may be cleavable, facilitating release of the cytotoxic drug in the cell. For example, acid-labile, peptidase-sensitive, dimethyl linker or disulfide-containing linkers (Chari et al., Cancer Research (1992) 52:127) may be used. Alternatively, various nonproteinaceous polymers, such polyethylene glycol (PEG), polypropylene glycol, polyoxyalkylenes, or copolymers of polyethylene glycol and polypropylene glycol, may find use as linkers.

Pharmaceutical compositions are also contemplated, where one or more therapeutically active agents are formulated. Formulations of the conjugates described herein may be prepared for storage by mixing a conjugate having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants or polyethylene glycol (PEG). In another embodiment, the pharmaceutical composition with the conjugate is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

Conjugate molecules disclosed herein may also be formulated as immunoliposomes. A liposome is a small vesicle comprising various types of lipids, phospholipids and/or surfactant that is useful for delivery of a therapeutic agent to a mammal. Liposomes containing the conjugates are prepared by methods known in the art. The liposome components are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome.

Conjugate molecules described herein may also be entrapped in microcapsules prepared by methods such as coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Sustained-release preparations may be prepared. Suitable examples of sustained-release preparations include semipermeable matrices of solid hydrophobic polymer, which matrices are in the form of shaped articles, e.g., films, or microcapsules. Examples of sustained-release matrices include polyesters, hydrogels (for example poly(2-hydroxyethyl-methacrylate), or poly(vinylalcohol)), polylactides, copolymers of L-glutamic acid and gamma ethyl-L-glutamate, non-degradable ethylene-vinyl acetate, degradable lactic acid-glycolic acid copolymers (which are injectable microspheres composed of lactic acid-glycolic acid copolymer and leuprolide acetate), and poly-D-(−)-3-hydroxybutyric acid) which is a microsphere-based delivery system composed of the desired bioactive molecule incorporated into a matrix of poly-DL-lactide-co-glycolide (PLG).

Conjugates may also be linked to nanoparticle surfaces using linking methods described herein. In one embodiment, the nanoparticles can be used for imaging or therapeutic purposes.

Administration of a pharmaceutical composition comprising a conjugate described herein, preferably in the form of a sterile aqueous solution, may be done in a variety of ways, including orally, subcutaneously, intravenously, intranasally, intraotically, transdermally, topically (e.g., gels, salves, lotions, creams), intraperitoneally, intramuscularly, intrapulmonary, vaginally, parenterally, rectally, or intraocularly. As is known in the art, a pharmaceutical composition may be formulated accordingly depending upon the manner of introduction.

As used herein, the singular forms “a,” “an,” and “the” include plural references unless the context clearly dictates otherwise. For example, the term “a molecule” can also include a plurality of molecules.

Proximity-based sortase-mediated expressed protein ligation systems offer a number of features that make it a very favorable approach for bioconjugation reactions. First, proximity-based sortase-mediated expressed protein ligation combines release of recombinant proteins from the affinity column and bioconjugation into a single step. This greatly simplifies the entire bioconjugation procedure, saving time, money, and complexity. Second, proximity-based sortase-mediated expressed protein ligation allows site-specific conjugation of cargo. Site-specific functionalization has been shown to be beneficial in a number of applications including the preparation of protein-drug conjugates, which often exhibit higher efficacy than randomly labeled targeting ligands. The site-specific attachment of targeting ligands to nanoparticles has also been shown to improve nanoparticle avidity. Third, proximity-based sortase-mediated expressed protein ligation conjugates the peptide-to-ligand in a 1:1 stoichiometric manner. This can be important when labeling targeting ligands with imaging agents, since it allows for precise quantitative imaging. It is also beneficial for characterizing nanoparticle bioconjugations. Fourth, the conditions used to release protein from the affinity column can be manipulated to ensure that essentially all recovered protein is conjugated with the desired cargo. This eliminates the often-difficult process of purifying conjugated products from unconjugated proteins. Since in many applications a large protein is labeled with low molecular weight drugs or imaging agents, the conjugated and unconjugated forms of the protein can differ by as little as a few hundred to a few thousand Da, potentially without a significant change to hydrophobicity or charge. A slight peptide excess is used to achieve complete ligation; however, excess peptide is easily removed via dialysis or gel chromatography. This purification step is analogous to the removal of, e.g., imidazole from His-tagged protein samples that have been affinity purified using a nickel column. Fifth, in contrast to STEPL, construction of the proximity-based sortase-mediated expressed protein ligation system as a two-construct, proximity-based sortase-mediated protein purification and ligation method can yield improved results over the single-step/single-construct method in which sortase is fused directly to the expressed protein. Although this latter method works well with shorter proteins, in some instances, the sortase interferes with the proper folding of larger or more complex proteins (e.g., scFv proteins), thereby disrupting the secondary structure of the protein of interest, and additionally, this approach may be incompatible with protein expression systems where calcium is present (e.g., yeast and mammalian systems).

In sum, described herein are flexible and efficient systems for molecular imaging and targeted therapeutics. Moreover, because they can be used to link virtually any bacterially expressible protein with any cargo that can be attached, e.g., a triglycine peptide, they have applications in many fields.

Any patent, patent application publication, or scientific publication, cited herein, is incorporated by reference herein in its entirety.

The following examples are presented to more fully illustrate preferred embodiments. They should in no way be construed, however, to limit the invention's broad scope.

### EXAMPLES

### Example 1: Proximity-Based Sortase-Mediated Protein Ligation (PBSL) Using Binding Partners From Lasso Peptide Biosynthesis Systems

In this Example, a bacterial sortase is used for targeting ligand purification and site-specific conjugation. The method uses two protein constructs, each including a binding sequence of one binding pair member, where the pair is a leader peptide and a B1 protein from a lasso peptide biosynthesis system.

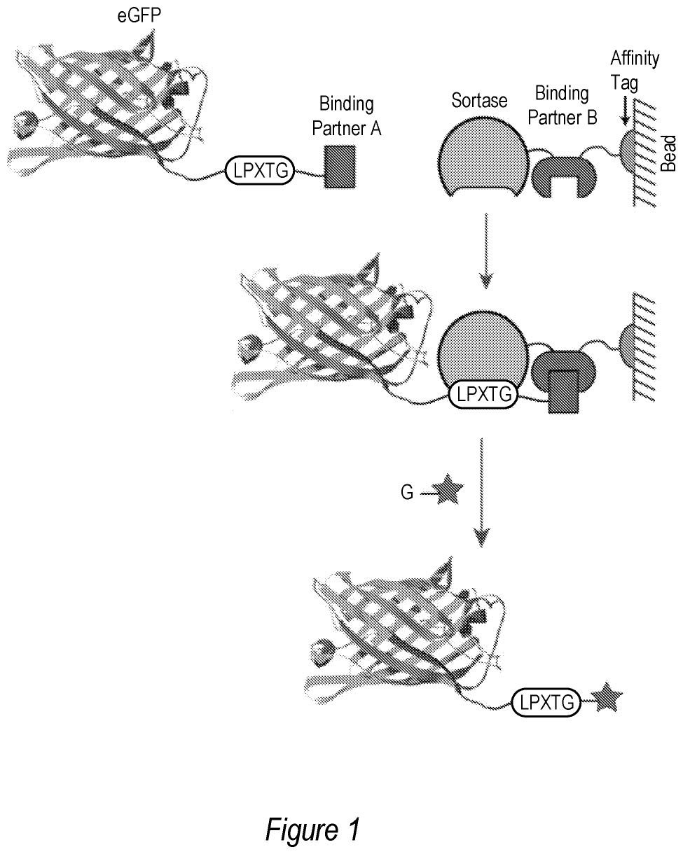

FIG. 1. General Scheme of Proximity-based Sortase Ligation on a Solid Support. A protein of interest is expressed in frame with a sortase recognition motif (e.g., LPXTG) and one binding pair member (Binding Partner A). In parallel, a sortase (Srt), a calcium-dependent transpeptidase, is expressed in frame with a second binding pair member (Binding Partner B) and an affinity tag. The sortase construct can be bound to an affinity column or beads. The protein is captured by the sortase construct via interaction of the binding pairs. The protein is subsequently released from the affinity column upon ligation to a peptide/protein with an N-terminal glycine. If desired, the peptide/protein can be labeled with a chemical or biological moiety (e.g., imaging agent, drug, click chemistry group, hapten, oligonucleotide, etc.), indicated as a star. Proximity-based sortase ligation can also be performed if sortase is expressed between the second binding pair member and the affinity tag.

Table 1. Representative leader peptide and B1 protein pairs from lasso peptide biosynthesis systems that can be used for PBSL. TfuA and TfuB are from Thermobifida fusca. LarA and LarE are from the biosynthesis system of lariatin produced by Rhodococcus jostii K01-B0171. StmA and StmE are from the biosynthesis system of streptomonomicin from Streptomonospora alba. PsmA and PsmB1 are from the lasso peptide synthesis gene cluster from Bacillus pseudomycoides. PadeA and PadeB1 are from the biosynthesis system for paeninodin produced by Paenibacillus dendritiformis C454.

TABLE 1

| | Leader peptide | B1 protein |
|---|---|---|
| TfuA | MEKKKYTAPQLAKVGEFK TfuB1 | METTGAEFRLRPEISVAQTDYGMVLLDGRSGEYWQL |
| | EATG (SEQ ID NO: 24) | NDTAALIVQRLLDGHSPADVAQFLTSEYEVERTDAE |
| | | RDIAALVTSLKENGMALP (SEQ ID NO: 25) |
| LarA | MTSQPSKKTYNAPSLVQR LarE | MVLRLRKNVIITPTEYGAVALDERSGDYYQLNSTAA |
| | GKFARTTA (SEQ ID NO: 26) | LILDQLTKKIPVESIAARIALDFEVSKAQASADLDE |
| | | YLRMLREQGLLR (SEQ ID NO: 27) |
| StmA | MSAYEIPTLTRIGKFKDV StmE | MAALMRVQMRPNVQIVSRDGDNFVLNLRNGEYWHLN |
| | TK (SEQ ID NO: 28) | SSAYTMLTRIADGETVDEVARDISAATSAPEQTVRD |
| | | DLTELVDQLRKAKLVEVRHS (SEQ ID NO: 29) |
| PsmA | MKQEWQSPVLEVLDINMT PsmB1 | MSSKQTISLHSFVVQGQENVVSDMDGEKVMMSIHNG |
| | M (SEQ ID NO: 30) | KYYNLGGIGGEIWSLINELISVNQVIDILLSRYMIE |
| | | EAECKEQVLSFLNHLYAGELISVDEKL (SEQ ID |
| | | NO: 31) |
| PadeA | MKKQYSKPSLEVLEVHOT PadeB1 | MSKLHSITPVDTLVQCEGHIVSDMAGEKVMLSVQKG |
| | M (SEQ ID NO: 32) | KYYNLGTLGGEIWDMLITPVKAEHIIQSILSEYEVE |
| | | SSECEEDILLFLSDLEHEGLIRHVKOR (SEQ ID |
| | | NO: 33) |

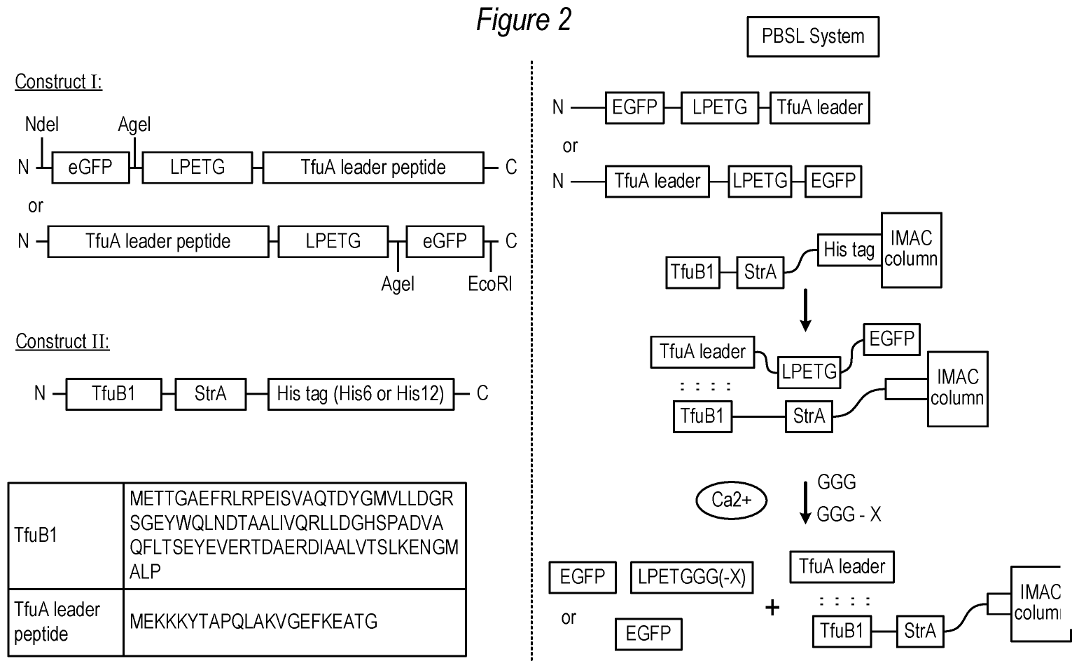

FIG. 2. TfuA leader-TfuB1 peptide-protein pair based PBSL design. Lasso peptides, a class of ribosomally synthesized and post-translationally modified peptides (RiPPs), exhibit a unique 3-D interlocked rotaxane structure and are found in diverse bacterial lineages. Such unique structure is introduced by lasso peptide synthetase encompassing proteins B and C. The B enzyme is split into two distinct proteins, B1 and B2. The B1 protein recognizes the leader sequence of the precursor peptide, the B2 protein cleaves it, and the C protein catalyzes the formation of the macrolactam ring. The B1 protein from the thermophilic actinobacteria, Thermobifida fusca (TfuB1) recognize the leader peptide (TfuA-Leader) and jointly form a hydrophobic patch with a K_{d} of ˜6 nM. Similar to SpyTag-SpyCatcher peptide-protein pair, the TfuA leader-TfuB1 peptide-protein pair can also be applied for proximity-based sortase-mediated ligation (PBSL). The TfuA-leader can be fused to either N-terminal of C-terminal of a target protein (e.g., EGFP), and then the expressed target protein can be captured non-covalently by an immobilized TfuB1-SrtA fusion protein during purification. Following the SrtA mediated ligation reaction with GGG, TfuA-leader peptide is cleaved off, the target protein is released. In addition, the C-terminal fusion of TfuA-leader peptide to the target protein allows addition of functional moieties (X), if desired, to the target protein through GGG ligation (GGG-X), simplifying target protein purification and labeling to a single step. Other peptide/protein pairs from analogous lasso peptide biosynthesis systems (see Table 1) can be used in a similar manner.

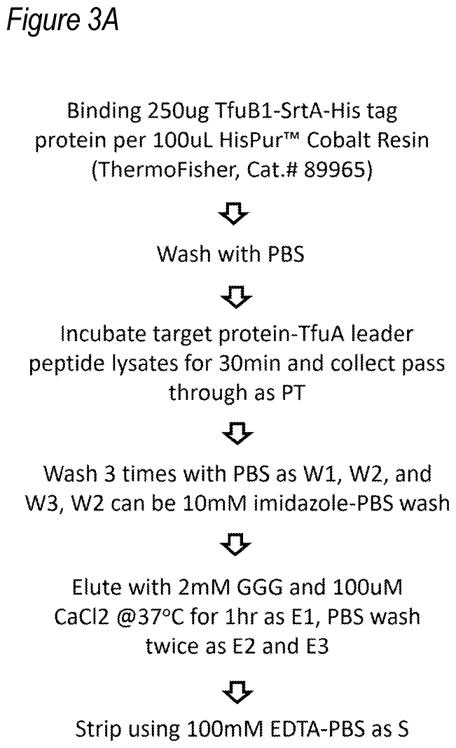

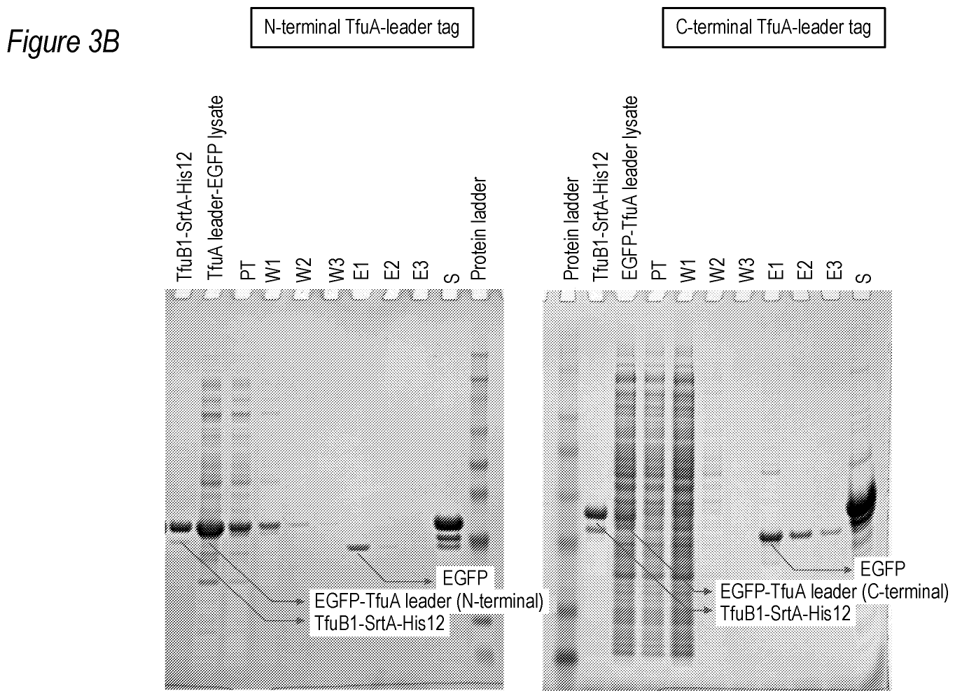

FIGS. 3A-3B. Target protein purification through TfuB1-TfuA leader protein-peptide pair based PBSL. (A) Flow diagram describing purification of target proteins via proximity-based sortase-mediated ligation (PBSL) using lasso leader peptide/protein binding pairs. Purified TfuB1-SrtA-His6 or TfuB1-SrtA-His12 protein is firstly immobilized onto IMAC column. The TfuA-leader peptide can be fused to either N-terminal of C-terminal of target protein (EGFP), and then the expressed target protein can be captured by TfuB1-SrtA fusion protein onto IMAC column during purification. Adding Ca²⁺ trigger the SrtA mediated ligation reaction with GGG (triglycine), TfuA-leader peptide is then cleaved off, the target protein is released and purified. (B) 4-12% SDS-PAGE showed the purification of target protein (EGFP) using TfuB1-TfuA leader protein-peptide paired PBSL. PT; pass through; W1: 1st wash; W2: 2nd wash; W3: 3rd wash; E1: 1st elution; E2: 2nd elution; E3: 3rd elution; S: Strip from column.

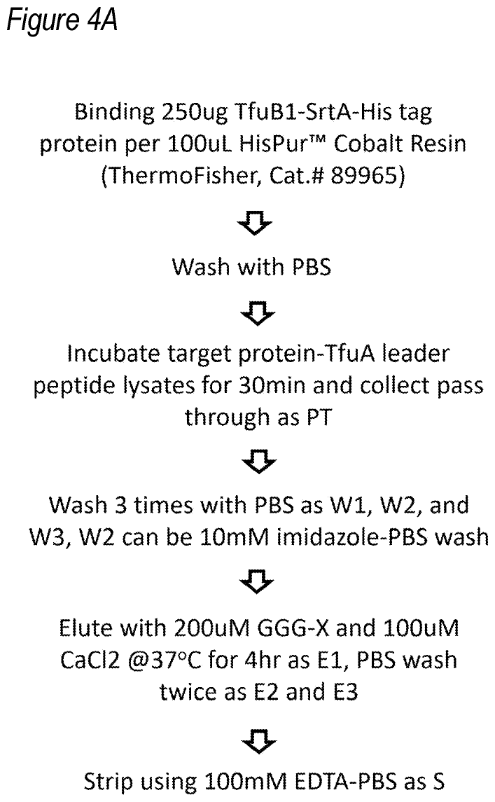

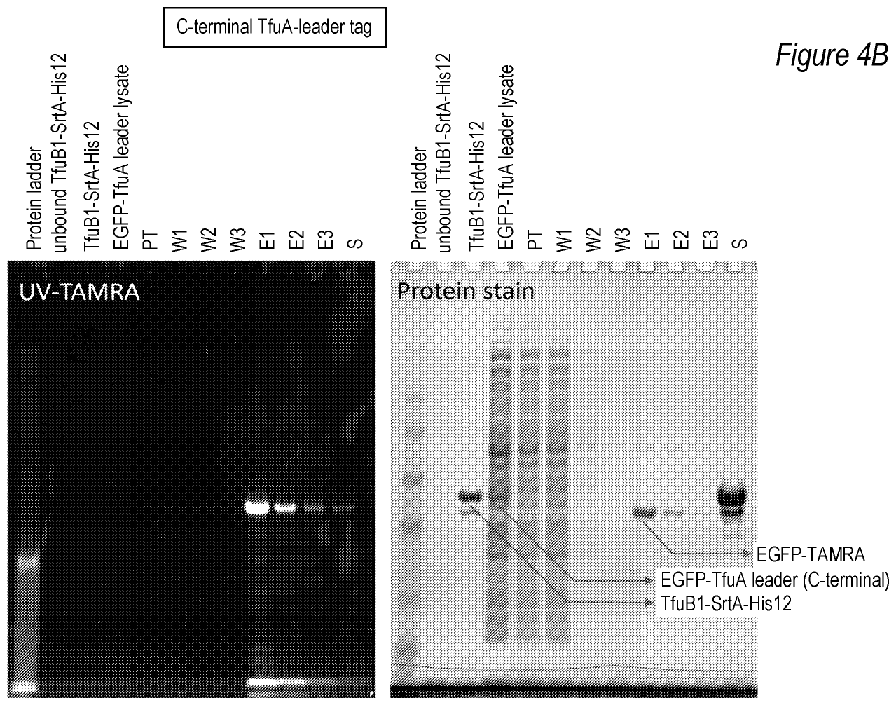

FIGS. 4A-4B. Target protein purification+labeling through TfuB1-TfuA leader protein-peptide pair based PBSL. (A) Flow diagram describing purification and labeling of target proteins via proximity-based sortase-mediated ligation (PBSL) using lasso leader peptide/protein binding pairs. C terminal fusion of TfuA-leader peptide to the target protein allows one-step target protein purification and labeling. Upon binding of the target protein (EGFP)-TfuA leader fusion onto IMAC column via TfuB1-TfuA leader interaction, Ca²⁺ can trigger the SrtA mediated ligation reaction with GGG bearing functional moieties, such as fluorescent dyes, chemical reaction groups, etc. TfuA-leader peptide is then cleaved off, the target protein is released with labeling. (B) 4-12% SDS-PAGE showed the purification and labeling of target protein (EGFP) with TAMRA dye using TfuB1-TfuA leader protein-peptide paired PBSL. PT: pass through; W1: 1st wash; W2: 2nd wash; W3: 3rd wash; E1; 1st elution; E2: 2nd elution; E3: 3rd elution; S: Strip from column.

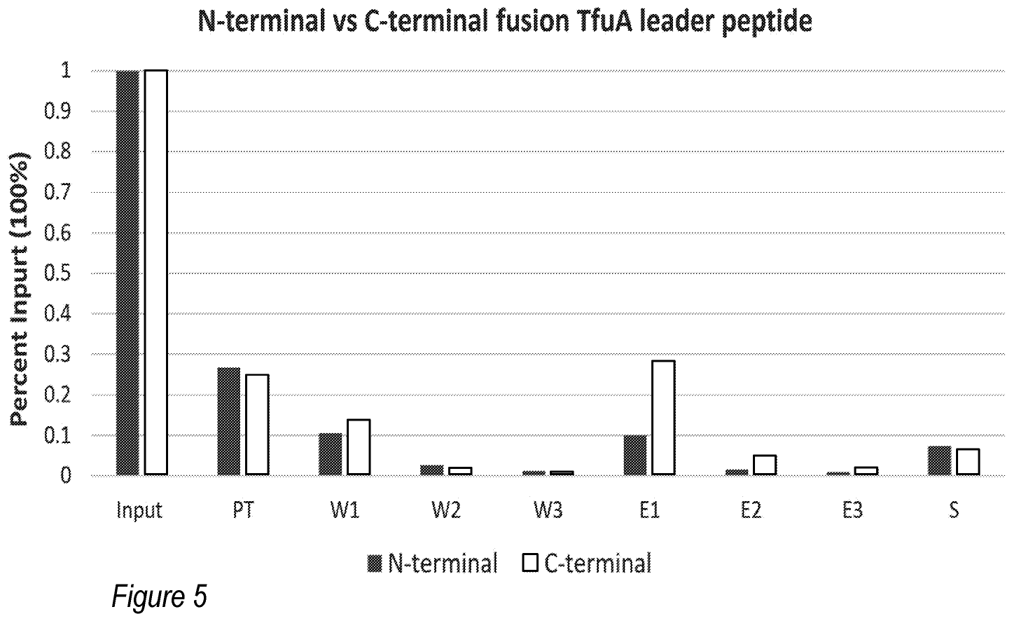

FIG. 5. Comparison of the target protein purification efficiency via PBSL using either an N-terminal or C-terminal fusion to a TfuA leader peptide. To compare the purification efficiency between TfuA leader peptide N-terminal fusion to the target protein (EGFP) and C-terminal fusion to the target protein (EGFP), the same amount of input target protein (EGFP) was used to go through PBSL purification procedures to assay the difference. Input: Input lysate; PT: pass through; W1: 1st wash; W2: 2nd wash; W3: 3rd wash; E1: 1st elution; E2: 2nd elution; E3: 3rd elution; S: Strip from column. The EGFP florescent intensity of samples collected from each step was measured. The amount of target protein in each step was reflected by the percent of the total EGFP fluorescent intensity in each step to total EGFP fluorescent intensity of the input. Conclusion: Compared to TfuA leader peptide C-terminal fusion to the target protein, N-terminal fusion showed similar TfuA leader peptide-TfuB1 protein binding. However, N-terminal fusion of TfuA leader peptide showed less cleavage and release of the target protein (10%-15% of the input) compared to C-ternmial fusion (˜30-40% of the input), there is more uncleaved construct left on the resin as shown in the stripped fraction(S).

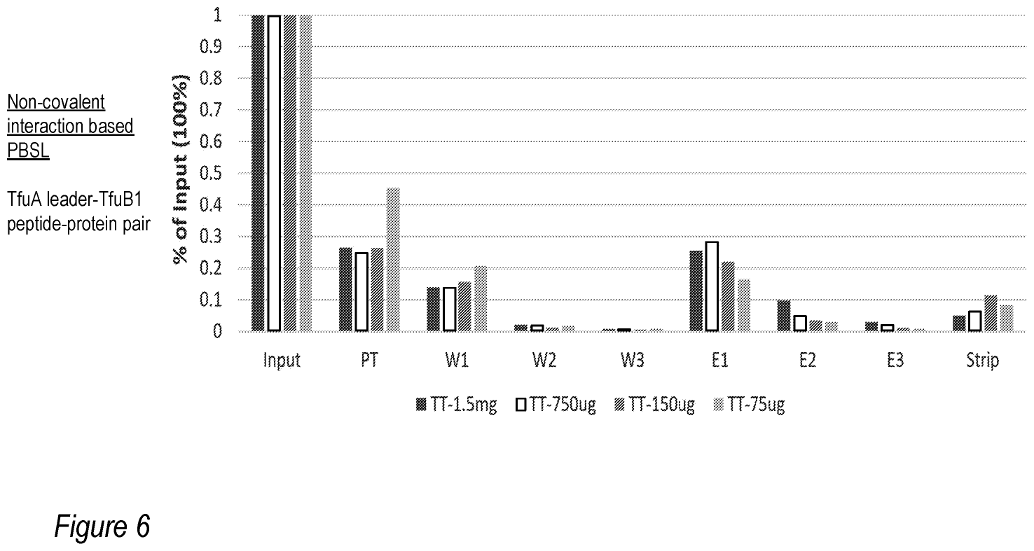

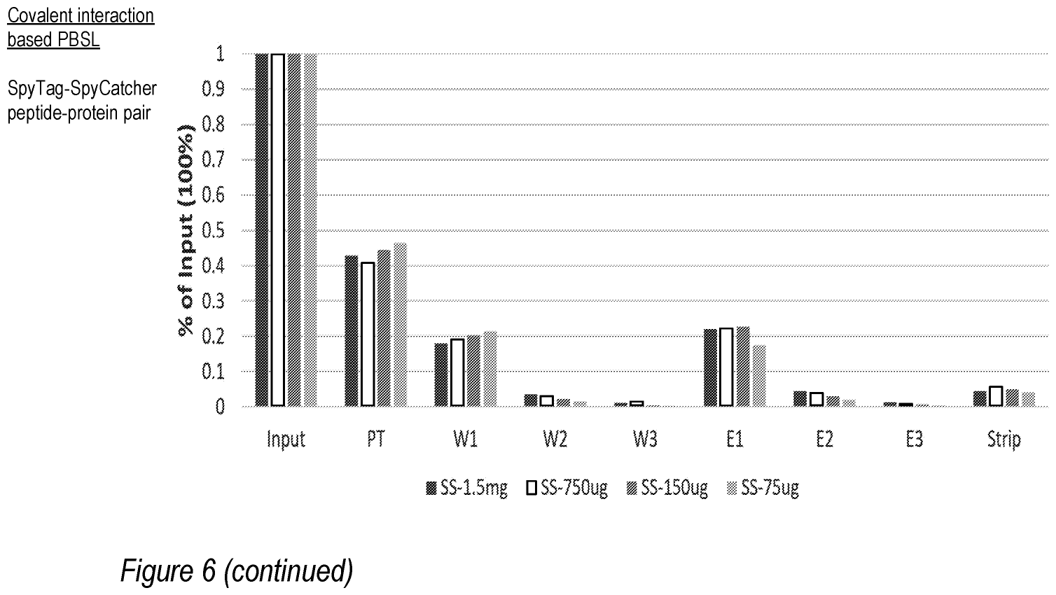

FIG. 6. Comparison of the target protein purification efficiency via PBSL using either Lasso leader peptide/protein pairs (non-covalent) or SpyCatcher/SpyTag protein pairs (covalent).

To compare the purification efficiency of non-covalent interaction based PBSL and covalent interaction based PBSL, the same amount of input target protein (EGFP) was used to go through each purification procedures to assay the difference. The SrtA fused capture protein, SpyCatcher or TfuB1, was immobilized on IMAC column by His tag with different loading amount: 1.5 mg/300 μL resin, 750 μg/300 μL resin, 150 μg/300 μL resin, and 75 μg/300 μL resin. For SpyTag-SpyCatcher system, EGFP-SpyTag lysates were incubated with resin for 30 min, allowing SpyTag and SpyCatcher to form a covalent bond. For TfuA leader-TfuB1 system, the capture of target protein is dependent on non-covalent interaction of the peptide-protein pair. After SrtA mediated ligation, the peptide tag remains on the column and the purified target protein (EGFP) is released. Input: Input lysate; PT: pass through; W1: 1st wash; W2: 2nd wash; W3: 3rd wash; E1: 1st elution; E2: 2nd elution; E3: 3rd elution; S: Strip from column. The EGFP florescent intensity of samples collected from each step was measured. The amount of target protein in each step was reflected by the percent of the total EGFP fluorescent intensity in each step to total EGFP fluorescent intensity of the input. Conclusion: Both systems showed comparable purification efficiency (˜30-40% of the input, under conditions utilized), and reached a plateau while increasing the capture protein loading amount on the resin.

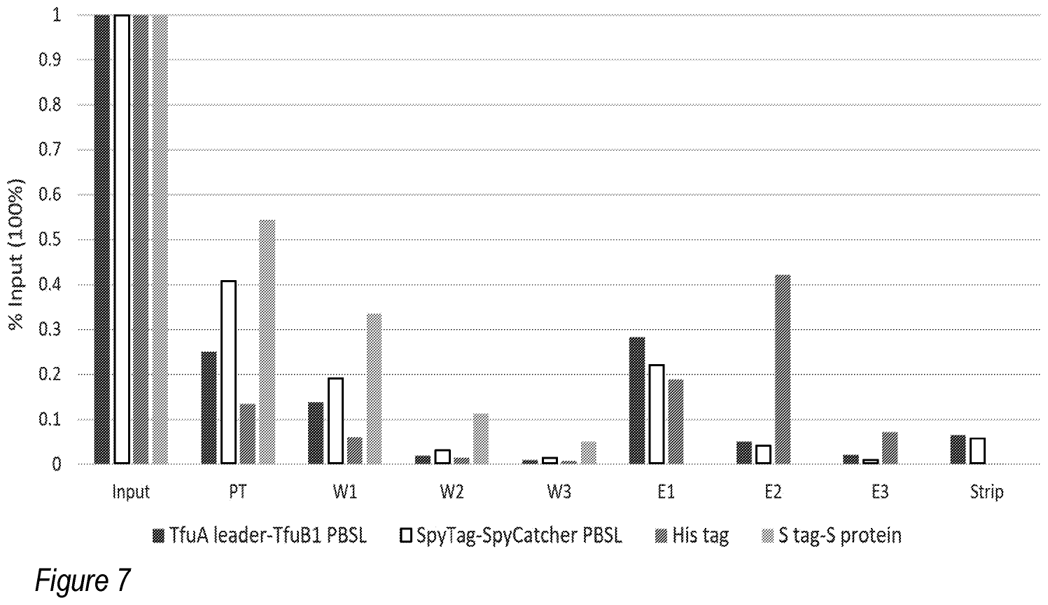

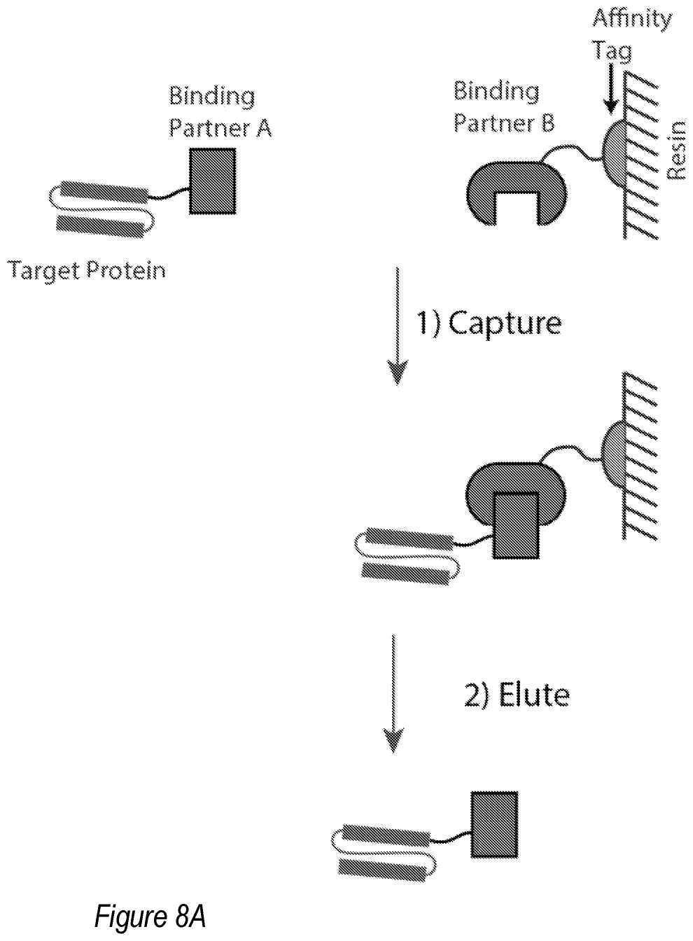

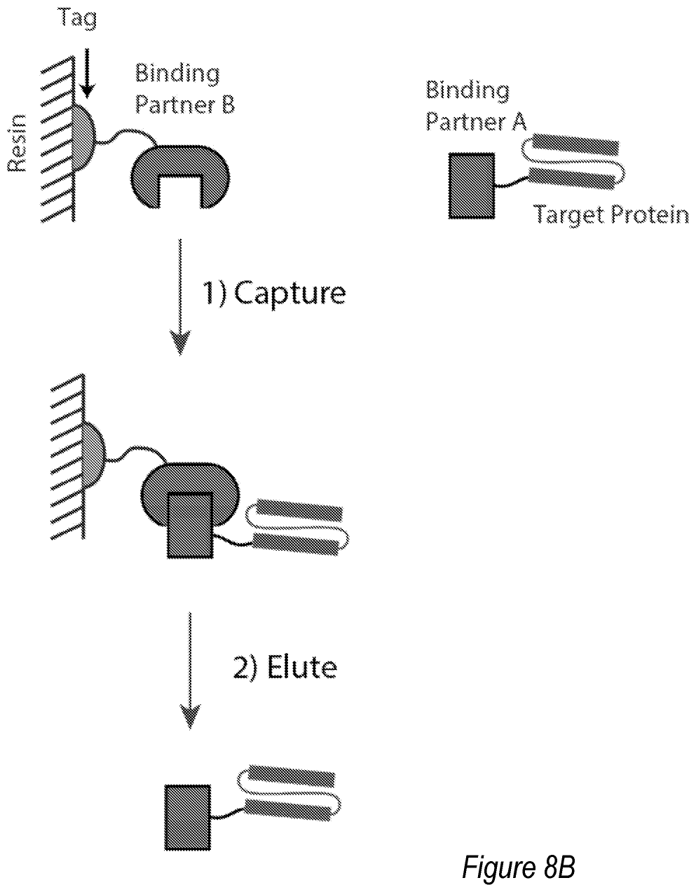

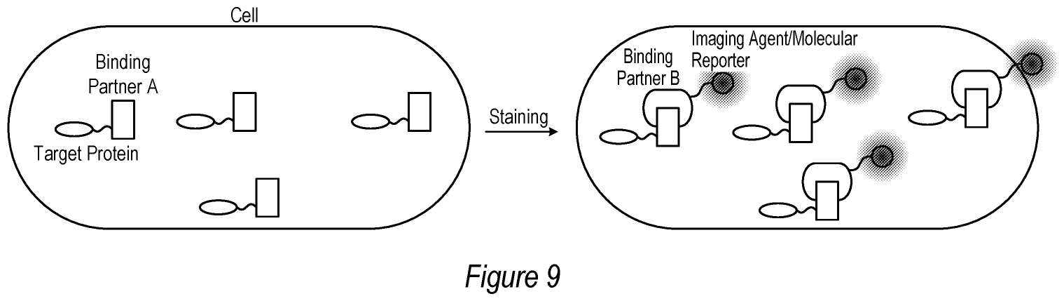

FIG. 7. Comparison of TfuA leader-TfuB1 based PBSL with other purification systems. In addition to comparing the lasso leader peptide/protein pair to SpyTag-SpyCatcher peptide-protein pair based PBSL, TfuA leader-TfuB 1 peptide-protein pair based PBSL was also compared with His tag protein purification system as well as S tag-S protein system in terms of protein purification efficiency. The same amount of total input protein, which is reflected as total EGFP fluorescent intensity, was applied to each purification system. Input: Input lysate; PT: pass through; W1: 1st wash; W2: 2nd wash; W3: 3rd wash; E1: 1st elution; E2: 2nd elution; E3: 3rd elution; S: Strip from column. The EGFP florescent intensity of samples collected from each step was measured. The amount of target protein in each step was reflected by the percent of the total EGFP fluorescent intensity in each step to total EGFP fluorescent intensity of the input. Conclusion: Both PBSL showed comparable purification efficiency (˜30-40% of the input) and 40-50% of input target protein loss. S tag-S protein showed about 50% input target protein binding, but it gradually lost more target protein during wash steps and did not achieve successful elution.

Those skilled in the art will appreciate that changes could be made to the embodiments described without departing from the broad inventive concept thereof. It is understood, therefore, that this invention is not limited to a particular disclosed embodiment but is intended to cover modifications within the invention's spirit and scope, as defined by the claims.

What is claimed is:

1. A conjugate protein composition comprising a first fusion protein and a second fusion protein, wherein:
(i) the first fusion protein comprises a protein of interest in series with a sortase recognition motif and a first binding pair member; wherein:
said protein of interest is N-terminal and connected via a linker to said sortase recognition motif and said sortase recognition motif is N-terminal and connected via a linker to the first binding pair member; or
said first binding pair member is N-terminal and connected via a linker to said sortase recognition motif and said sortase recognition motif is N-terminal and connected via a linker to said protein of interest; and
(ii) the second fusion protein comprises a second binding pair member in series with a sortase and optionally a first affinity tag having a selective affinity for a first affinity tag resin, wherein:
said second binding pair member is N-terminal and connected via a linker to said sortase; or
said sortase is N-terminal and connected via a linker to said second binding pair member;
and when said first affinity tag is present, said first affinity tag is connected via a linker either N-terminal or C-terminal to said second binding pair member in series with said sortase;
wherein the binding pair members comprise a leader peptide and a B1 protein pair from a lasso peptide biosynthesis system that can form a heterodimer.

2. The conjugate protein composition of claim 1, wherein said first binding pair member is the leader peptide from the lasso peptide biosynthesis system.

3. The conjugate protein composition of claim 2, wherein the leader peptide is located at the C-terminus of the first fusion protein.

4. The conjugate protein composition of claim 1, wherein the binding pair members comprise a plurality of the leader peptides in tandem and/or a plurality of the B1 proteins in tandem.

5. The conjugate protein composition of any one of claims 1-4, wherein the leader peptide and the B1 protein pair are selected from (i) the leader peptide TfuA and B1 protein TfuB 1 from Thermobifida fusca; (ii) the leader peptide LarA and B1 protein LarE from Rhodococcus jostii K01-B0171; (iii) the leader peptide StmA and B1 protein StmE from Streptomonospora alba; (iv) the leader peptide PsmA and B1 protein PsmB1 from Bacillus pseudomycoides; and (v) the leader peptide PadeA and B1 protein PadeB 1 from Paenibacillus dendritiformis C454.

6. The conjugate protein composition of claim 5, wherein the leader peptide and B 1 protein pair are TfuA and TfuB1, respectively.

7. The conjugate protein composition of any one of the preceding claims, wherein the first affinity tag is selected from the group consisting of a histidine tag (His tag), a chitin-binding domain, a calmodulin tag, a polyglutamate tag, a maltose binding protein, glutathione-S-transferase, an S-tag, SBP-tag, Strep-tag, Strep-tag II, green fluorescent protein-tag, thioredoxin tag, Nus-tag, Fc-tag, Halo-tag, FLAG-tag, V5-tag, VSV-tag, Xpress tag, E-tag, Myc-tag, HA-tag, Softag, and NE-tag, biotin (via biotin ligase), BirA, AviTag, BCCP, SpyTag, SpyCathcher, SnoopTag, and SnoopCatcher.

8. The conjugate protein composition of any one of claims 1-6, wherein the first affinity tag is absent and said second fusion protein is chemically conjugated to a resin.

9. The conjugate protein composition of any one of the preceding claims, wherein said first fusion protein further comprises a second affinity tag having a selective affinity for a second affinity tag resin.

10. The conjugate protein composition of claim 9, wherein the second affinity tag is selected from the group consisting of a histidine tag (His tag), a chitin-binding domain, a calmodulin tag, a polyglutamate tag, a maltose bidning protein, glutathione-S-transferase, an S-tag, SBP-tag, Strep-tag, Strep-tag II, green fluorescent protein-tag, thioredoxin tag, Nus-tag, Fc-tag, Halo-tag, FLAG-tag, V5-tag, VSV-tag, Xpress tag, E-tag, Myc-tag, HA-tag, Softag, and NE-tag, biotin (via biotin ligase), BirA, AviTag, BCCP, SpyTag, SpyCathcher, SnoopTag, and SnoopCatcher.

11. The conjugate protein composition of any one of the preceding claims, wherein said sortase is selected from the group consisting of sortase A (SrtA), sortase B (SrtB), sortase C (SrtC), sortase D (SrtD), sortase E (SrtE), sortase F (SrtF), and variants thereof.

12. The conjugate protein composition of any one of the preceding claims, wherein the sortase recognition motif is selected from the group consisting of LPXTG (SEQ ID NO: 1), LPKTG (SEQ ID NO: 2), LPATG (SEQ ID NO: 3), LPNTG (SEQ ID NO: 4), LPETG (SEQ ID NO: 5), LPXAG (SEQ ID NO: 6), LPNAG (SEQ ID NO: 7), LPXTA (SEQ ID NO: 8), LPNTA (SEQ ID NO: 9), LGXTG (SEQ ID NO: 10), LGATG (SEQ ID NO: 11), IPXTG (SEQ ID NO: 12), IPNTG (SEQ ID NO: 13), IPETG (SEQ ID NO: 14), and NPQTN (SEQ ID NO: 15), LAXTG (SEQ ID NO: 16), LPXSG (SEQ ID NO: 17), LSETG (SEQ ID NO: 18), LPXCG (SEQ ID NO: 19), LPXAG (SEQ ID NO: 20), and XPETG (SEQ ID NO: 21).

13. The conjugate protein composition of any one of the preceding claims, wherein the linkers are flexible GS-rich linkers.

14. The conjugate protein composition of claim 13, wherein the flexible GS-rich linkers are (GGS)ₙ linkers, where n is an integer.

15. The conjugate protein composition of claim 14, wherein the flexible GS-rich linkers have from 3 to 5 GGS repeats.

16. The conjugate protein composition of any one of the preceding claims, further comprising said first affinity tag resin to which said first affinity tag selectively binds.

17. The conjugate protein composition of any one of the preceding claims, wherein said protein of interest is an antibody or antigen-binding fragment thereof.

18. The conjugate protein composition of claim 17, wherein the antibody or antigen-binding fragment thereof comprises an immunoglobulin G (IgG), an immunoglobulin M (IgM), an immunoglobulin D (IgD), an immunoglobulin E (IgE), or an immunoglobulin A (IgA).

19. The conjugate protein composition of claim 17, wherein the antibody or antigen-binding fragment thereof comprises an Fv, Fab, Fab′, (Fab′)2 domain, single-chain antibody fragment, or a fusion thereof.

20. A vector encoding said first fusion protein and/or second fusion protein of any one of claims 1-19.

21. A cell for recombinantly expressing the first fusion protein and/or second fusion protein of any one of claims 1-19, wherein said cell is a bacterial cell, a yeast cell, an insect cell, or a mammalian cell.

22. A conjugation method, said method comprising;
(a) providing the first and the second fusion proteins from the protein conjugate composition according to any one of claims 1-19;
(b) contacting the first fusion protein with the second fusion protein under conditions where the leader peptide forms a heterodimer with the B1 protein; and
(c) adding calcium and glycine or a peptide or protein with an N-terminal glycine, under conditions where the sortase catalyzes conjugation and release of the protein of interest conjugated to said glycine or said peptide or protein with an N-terminal glycine.

23. The conjugation method of claim 22, wherein said protein of interest is an antibody of interest or an antigen-binding fragment thereof.

24. The conjugation method of claim 23, wherein said sortase recognition motif is C-terminal to at least one heavy chain or light chain of said antibody.

25. The conjugation method of any one of claims 22-24, wherein said glycine or said peptide or protein with an N-terminal glycine further comprises a functional group.

26. The conjugation method of any one of claims 22-24, wherein said glycine or said peptide or protein with an N-terminal glycine is fused or linked to a protein, an enzyme, a drug molecule, an antibiotic, an imaging agent, a fluorescent dye, a metal chelate, a chelated metal, a polymer, a polyethylene glycol, an oligonucleotide, a photocrosslinker, a click chemistry group, an alkyne, an azide, a hapten, a biotin, a protein, a small molecule, azodibenzocyclooctyne (ADIBO), digoxigenin (DIG), dibenzocyclooctyne (DBCO), trans-cyclooctene (TCO), tetrazine, or a nanoparticle.

27. The conjugation method of any one of claims 22-26, wherein said glycine is at the N-terminus of said protein of interest and forms a cyclized protein of interest.

28. A method for purifying a protein of interest, said method comprising;
(a) providing the first and the second fusion proteins from the protein conjugate composition of any one of claims 1-19;
(b) contacting the first fusion protein with the second fusion protein under conditions where the leader peptide forms a heterodimer with the B1 protein;
(c) adding calcium alone or calcium and glycine, or calcium and a peptide or protein with an N-terminal glycine, under conditions where the sortase catalyzes respectively, (i) release of the protein of interest or (ii) conjugation and release of the protein of interest conjugated to said glycine or said peptide or protein with an N-terminal glycine; and
(d) separating said protein of interest or said conjugated protein of interest.

29. The method of claim 28, wherein said first affinity tag is selectively bound to said first affinity tag resin prior to the step of contacting said first fusion protein with said second fusion protein.

30. The method of claim 28, wherein said first affinity tag is selectively bound to said first affinity tag resin after the step of contacting said first fusion protein with said second fusion protein, but before step (c).

31. The method of any one of claims 28-30, wherein said first protein further comprises a second affinity tag having a selective affinity for a second affinity tag resin.

32. The method of claim 31, wherein said second affinity tag is selectively bound to said second affinity tag resin prior to the step of contacting said first fusion protein with said second fusion protein.

33. The method of claim 31, wherein said second affinity tag is selectively bound to said second affinity tag resin after the step of contacting said first fusion protein with said second fusion protein, but before step (c).

34. The method of claim 31, wherein said second affinity tag is selectively bound to said second affinity tag resin after step (c).

35. The method of any one of claims 28-34, wherein said first and said second affinity tags are each selected from the group consisting of a histidine tag (His tag), a chitin-binding domain, a calmodulin tag, a polyglutamate tag, a maltose bidning protein, glutathione-S-transferase, an S-tag, SBP-tag, Strep-tag, Strep-tag II, green fluorescent protein-tag, thioredoxin tag, Nus-tag, Fc-tag, Halo-tag, FLAG-tag, V5-tag, VSV-tag, Xpress tag, E-tag, Myc-tag, HA-tag, Softag, and NE-tag, biotin (via biotin ligase), BirA, AviTag, BCCP, SpyTag, SpyCathcher, SnoopTag, and SnoopCatcher.

36. A conjugate protein composition comprising a first fusion protein and a second fusion protein, wherein:
(i) the first fusion protein comprises a protein of interest in series with and connected via a linker to a first binding pair member; and
(ii) the second fusion protein comprises a second binding pair member optionally in series with and connected via a linker to a first affinity tag having a selective affinity for a first affinity tag resin;
wherein the binding pair members comprise a leader peptide and a B1 protein pair from a lasso peptide biosynthesis system that can form a heterodimer.

37. The conjugate protein composition of claim 36, wherein said first binding pair member is the leader peptide from the lasso peptide biosynthesis system.

38. The conjugate protein composition of claim 37, wherein the leader peptide is located at the C-terminus of the first fusion protein.

39. The conjugate protein composition of claim 36, wherein the binding pair members comprise a plurality of the leader peptides in tandem and/or a plurality of the B1 proteins in tandem.

40. The conjugate protein composition of claim 36, wherein the first fusion protein contains a protease cleavage site between the protein of interest and the first binding pair member.

41. The conjugate protein composition of any one of claims 36-40, wherein the leader peptide and the B1 protein pair are selected from (i) the leader peptide TfuA and B1 protein TfuB1 from Thermobifida fusca; (ii) the leader peptide LarA and B1 protein LarE from Rhodococcus jostii K01-B0171; (iii) the leader peptide StmA and B 1 protein StmE from Streptomonospora alba; (iv) the leader peptide PsmA and B1 protein PsmB1 from Bacillus pseudomycoides; and (v) the leader peptide PadeA and B1 protein PadeB1 from Paenibacillus dendritiformis C454.

42. The conjugate protein composition of claims 41, wherein the leader peptide and B1 protein pair are TfuA and TfuB1, respectively.

43. The conjugate protein composition of any one of claims 36-42, wherein the first affinity tag is selected from the group consisting of a histidine tag (His tag), a chitin-binding domain, a calmodulin tag, a polyglutamate tag, a maltose binding protein, glutathione-S-transferase, an S-tag, SBP-tag, Strep-tag, Strep-tag II, green fluorescent protein-tag, thioredoxin tag, Nus-tag, Fc-tag, Halo-tag, FLAG-tag, V5-tag, VSV-tag, Xpress tag, E-tag, Myc-tag, HA-tag, Softag, and NE-tag, biotin (via biotin ligase), BirA, AviTag, BCCP, SpyTag, SpyCathcher, SnoopTag, and SnoopCatcher.

44. The conjugate protein composition of any one of claims 36-42, wherein the first affinity tag is absent and said second fusion protein is chemically conjugated to a resin.

45. The conjugate protein composition of any one of claims 36-44, wherein said first fusion protein further comprises a second affinity tag having a selective affinity for a second affinity tag resin.

46. The conjugate protein composition of claim 45, wherein the second affinity tag is selected from the group consisting of a histidine tag (His tag), a chitin-binding domain, a calmodulin tag, a polyglutamate tag, a maltose bidning protein, glutathione-S-transferase, an S-tag, SBP-tag, Strep-tag, Strep-tag II, green fluorescent protein-tag, thioredoxin tag, Nus-tag, Fc-tag, Halo-tag, FLAG-tag, V5-tag, VSV-tag, Xpress tag, E-tag, Myc-tag, HA-tag, Softag, and NE-tag, biotin (via biotin ligase), BirA, AviTag, BCCP, SpyTag, SpyCathcher, SnoopTag, and SnoopCatcher.

47. The conjugate protein composition of any one of claims 36-46, wherein the linkers are flexible GS-rich linkers.

48. The conjugate protein composition of claim 47, wherein the flexible GS-rich linkers are (GGS) n linkers, where n is an integer.

49. The conjugate protein composition of claim 48, wherein the flexible GS-rich linkers have from 3 to 5 GGS repeats.

50. The conjugate protein composition of any one of claims 36-43 and 45-49, further comprising said first affinity tag resin to which said first affinity tag selectively binds.

51. The conjugate protein composition of any one of claims 36-50, wherein said protein of interest is an antibody or antigen-binding fragment thereof.

52. The conjugate protein composition of claim 51, wherein the antibody or antigen-binding fragment thereof comprises an immunoglobulin G (IgG), an immunoglobulin M (IgM), an immunoglobulin D (IgD), an immunoglobulin E (IgE), or an immunoglobulin A (IgA).

53. The conjugate protein composition of claim 51, wherein the antibody or antigen-binding fragment thereof comprises an Fv, Fab, Fab′, (Fab′)2 domain, single-chain antibody fragment, or a fusion thereof.

54. A vector encoding said first fusion protein and/or the second fusion protein of any one of claims 36-54.

55. A cell for recombinantly expressing the first fusion protein and/or the second fusion protein of any one of claims 36-54, wherein said cell is a bacterial cell, a yeast cell, an insect cell, or a mammalian cell.

56. A method of purifying a protein of interest, said method comprising;
(a) providing the first and the second fusion proteins from the protein conjugate composition according to any one of claims 36-54;
(b) contacting the first fusion protein with the second fusion protein under conditions where the leader peptide forms a heterodimer with the B1 protein;
(c) eluting, dissociating, or cleaving the protein of interest from the second fusion protein; and
(d) isolating said protein of interest.

57. The method of claim 56, wherein said protein of interest is an antibody of interest or an antigen-binding fragment thereof.

58. The method of claim 56, wherein said first affinity tag is selectively bound to said first affinity tag resin prior to the step of contacting said first fusion protein with said second fusion protein.

59. The method of claim 56, wherein said first affinity tag is selectively bound to said first affinity tag resin after the step of contacting said first fusion protein with said second fusion protein, but before step (c).

60. The method of any one of claims 56-59, wherein said first protein further comprises a second affinity tag having a selective affinity for a second affinity tag resin.

61. The method of claim 60, wherein said second affinity tag is selectively bound to said second affinity tag resin prior to the step of contacting said first fusion protein with said second fusion protein.

62. The method of claim 60, wherein said second affinity tag is selectively bound to said second affinity tag resin after the step of contacting said first fusion protein with said second fusion protein, but before step (c).

63. The method of claim 60, wherein said second affinity tag is selectively bound to said second affinity tag resin after step (c).

64. The method of any one of claims 56-63, wherein said first and said second affinity tags are each selected from the group consisting of a histidine tag (His tag), a chitin-binding domain, a calmodulin tag, a polyglutamate tag, a maltose bidning protein, glutathione-S-transferase, an S-tag, SBP-tag, Strep-tag, Strep-tag II, green fluorescent protein-tag, thioredoxin tag, Nus-tag, Fc-tag, Halo-tag, FLAG-tag, V5-tag, VSV-tag, Xpress tag, E-tag, Myc-tag, HA-tag, Softag, and NE-tag, biotin (via biotin ligase), BirA, AviTag, BCCP, SpyTag, SpyCathcher, SnoopTag, and SnoopCatcher.

65. The method of claim 56, wherein the protein of interest is eluted, dissociated, or cleaved from the second fusion protein by using an eluting agent, low pH, heat or a protease.

66. A method of detecting a protein of interest, said method comprising;
(a) providing a protein of interest connected via a linker to a first binding pair member;
(b) contacting the first binding pair member with a second binding pair member wherein the second binding pair member is attached to an imaging agent or molecular reporter; and
(c) imaging or detecting said imaging agent or molecular reporter. wherein the first and second binding pair members comprise a leader peptide and a B1 protein pair from a lasso peptide biosynthesis system that can form a heterodimer.

67. The method of claim 66, wherein the protein of interest connected via a linker to a binding pair member is a fusion protein.

68. The method of claim 67, wherein the fusion protein is expressed in a cell, or the cellular membrane or secreted from the cell.

69. The method of claim 66, wherein the protein of interest is an antibody or antigen-binding fragment thereof.

70. The method of claim 69, wherein the antibody or antigen-binding fragment thereof comprises an immunoglobulin G (IgG), an immunoglobulin M (IgM), an immunoglobulin D (IgD), an immunoglobulin E (IgE), or an immunoglobulin A (IgA).

71. The method of claim 69, wherein the antibody or antigen-binding fragment thereof comprises an Fv, Fab, Fab′, (Fab′)2 domain, single-chain antibody fragment, or a fusion thereof.

72. The method of claim 66, wherein said molecular reporter is a fluorescent protein, a luciferase, horseradish peroxidase, alkaline phosphatase, glucose oxidase, or glucosidase.
